(19) United States
(12) Patent Application Publication
(10) Pub. No.: US 2015/0018563 A1
(43) Pub. Date: Jan. 15, 2015

(54) Surfactants and Solvents Containing Diels-Alder Adducts

(71) Applicant: Stepan Company, Northfield, IL (US)

(72) Inventors: Rick Tabor, Plymouth, MI (US); Randal J. Bernhardt, Antioch, IL (US); Franz J. Luxem, Palatine, IL (US); Chunhua Yao, Carmel, IN (US); Gregory James Wallace, Chicago, IL (US)

(21) Appl. No.: 14/498,231

(22) Filed: Sep. 26, 2014

Related U.S. Application Data
(63) Continuation of application No. PCT/US2013/034113, filed on Mar. 27, 2013.
(60) Provisional application No. 61/615,949, filed on Mar. 27, 2012.

(51) Int. Cl. C07D 209/48; C07C 303/04; C07C 305/16

(52) U.S. Cl. CPC C07D 209/48; C07C 305/16; C07C 303/04; C07C 2101/16
USPC 548/478; 558/32

(57) ABSTRACT

Surfactants and solvents containing derivatized adducts formed from Diels-Alder reactions of terpenes and unsaturated carboxylic acids or their derivatives are disclosed. Processes for making and derivatizing the Diels-Alder adducts are also disclosed.

This application claims priority to, and is a continuation of, International application No. PCT/US2013/034113 (International Publication No. WO 2013/148842), having an International filing date of Mar. 27, 2013. The PCT application claims priority to and claims benefit from U.S. provisional patent application No. 61/615,949, filed Mar. 27, 2012. The entire specifications of the PCT and provisional applications referred to above are hereby incorporated by reference.

### FIELD OF THE INVENTION

The presently described technology relates generally to surfactants and solvents containing Diels-Alder adducts of farnesene or myrcene, methods of derivatizing such adducts to form surfactants and solvents, and compositions comprising or incorporating such surfactants or solvents.

### BACKGROUND OF THE INVENTION

Surfactants and solvents are used in a wide variety of products such as household, industrial and institutional cleaning products. Desirable attributes for such products include the ability to emulsify, suspend or penetrate greasy or oily soils and suspend or disperse particulates in order to clean surfaces; and then prevent the soils, grease or particulates from redepositing on the newly cleaned surfaces. For example, a laundry detergent product should desirably remove dirt from clothes and then keep the dirt in suspended solution so that it is removed with the wash water instead of re-depositing on the washed clothes. For hard surface cleaners, it is desirable to have the ability to wet various surface types and couple or suspend soils to leave the surface free from residue in the form of streaking and/or filming.

Surfactants are also used in agricultural formulations to emulsify, suspend, liquefy and compatibilize active ingredients and enhance wetting to improve the delivery and efficacy of the active ingredient.

It is highly desirable that surfactants and solvents be biodegradable and obtained from biorenewable materials so that the end-use products employing such surfactants or solvents are more environmentally friendly. It is also desirable to prepare surfactants and solvents from low-cost feedstocks that are biorenewable and sustainable.

It has now been found that novel surfactants useful in the formulation of a variety of end use products can be obtained from derivatized Diels-Alder adducts of farnesene or myrcene and at least one dienophile.

### SUMMARY OF THE INVENTION

The present technology relates to surfactants prepared from Diels-Alder adducts of farnesene or myrcene that are further derivatized to form anionic, cationic, amine oxide, amphoteric or nonionic surfactants.

The present technology also relates to Diels-Alder adducts of farnesene or myrcene that are further derivatized to form useful solvents. The present technology further relates to methods of derivatizing the Diels-Alder adducts to form the surfactants and solvents.

In some embodiments, the Diels-Alder adducts are prepared by reacting farnesene or myrcene with an unsaturated carboxylic acid or its ester or anhydride. In other embodiments, the Diels-Alder adducts are prepared by reacting farnesene or myrcene with an unsaturated nitrile. The Diels-Alder adducts are then derivatized in a variety of ways to form the anionic, cationic, amine oxide, amphoteric or nonionic surfactants. In some embodiments, the Diels-Alder adducts are reacted with monomethyl polyethylene glycol or monomethyl triethylene glycol to form nonionic surfactants comprising mono- or di-esters. In other embodiments, the Diels-Alder adducts are reacted with an amine followed by oxidation to form amine oxide surfactants comprising amine oxides of the Diels-Alder adducts. In further embodiments, the Diels-Alder adducts are reacted with aromatic-substituted alcohols, followed by hydrogenation and sulfonation to form anionic surfactants comprising sulfonated adducts. In still further embodiments, the Diels-Alder adducts are reacted with a sugar to form nonionic surfactants comprising sugar esters of the adducts. In other embodiments, the Diels-Alder adducts are hydrogenated and further reacted with methanol and methyl or benzyl chloride, epichlorohydrin or dimethyl sulfate to form cationic surfactants comprising adducts containing an ammonium group. In other embodiments, farnesene or myrcene are reacted with a previously derivatized dienophile to form a surfactant, solvent or surfactant precursor which may be further derivatized to form a surfactant. In other embodiments, the Diels-Alder adducts are alkoxylated with ethylene oxide, propylene oxide or butylene oxide to form surfactants or solvents. In other embodiments, the Diels-Alder adducts are reacted with a mono- or oligo-ethyleneamine, or aminoethyl ethanolamine or mixtures thereof to form an imidazole, followed by quaternization with a suitable quaternizing agent such as an alkyl halide, an alkylaryl halide, epichlorohydrin or dimethyl sulfate to form quaternium surfactants. In other embodiments, the Diels-Alder adducts containing esters, anhydrides or carboxylic acids are derivatized by amidation with an alkyl amine or dialkyl amine followed by hydrogenation, followed by oxidation to form amine oxides. In other embodiments, the Diels-Alder adducts are hydrogenated, then sulfated, sulfonated or phosphated. In other embodiments, the Diels Alder adducts of farnesene or myrcene or mixtures thereof may be reacted directly with a sulfonating agent such as, for example, sodium or potassium bisulfite. In still further embodiments, the Diels-Alder adducts are reacted with an aromatic substituted alcohol or an amide to form a solvent.

### BRIEF DESCRIPTION OF SEVERAL VIEWS OF THE DRAWINGS

FIG. 1 is a graph comparing the cleaning performance of a composition of the present technology with that of conventional amine oxides.

### DETAILED DESCRIPTION OF THE INVENTION

The present technology provides a new approach for incorporating biorenewable material into surfactants and solvents. This approach comprises preparing a Diels-Alder adduct made from a biorenewable material and further derivatizing the Diels-Alder adduct to prepare surfactants and solvents that are useful in a wide variety of applications.

The surfactants and solvents of the present technology are prepared by reacting (a) biorenewable farnesene or myrcene with (b) a dienophile to form a Diels-Alder adduct, and then derivatizing the Diels-Alder adduct to form anionic, cationic, amine oxide, amphoteric, or nonionic surfactants or solvents. Alternatively, the surfactants and solvents of the present technology are prepared by derivatizing a dienophile and then reacting the derivatized dienophile with farnesene or myrcene to form a derivatized Diels-Alder adduct.

The biorenewable material is selected from terpenes. A requirement of the biorenewable material is that it functions as the diene component in a Diels-Alder reaction. Terpenes are composed of isoprene units and are classified according to the number of isoprene units in the molecule. Hemiterpenes comprise a single isoprene unit and isoprene derivatives. Isoprene or terpene derivatives are those that have been modified chemically, such as by oxidation or by rearrangement of the carbon skeleton. Monoterpenes comprise two isoprene units, sesquiterpenes comprise three isoprene units, triterpenes comprise six isoprene units and polyterpenes comprise long chains of many isoprene units. Suitable terpenes for use as the biorenewable material include myrcene and farnesene. Mixtures of these terpenes are also suitable.

Farnesene refers to a group of a biorenewable sesquiterpene chemical compounds that occur in nature and is a particularly preferred terpene for use herein. Farnesene is found in the coating of apples and other fruits, for example, and is thought to be responsible for the characteristic green apple color. A commercial source for farnesene is Amyris Inc. (Emeryville, Calif.).

The set of chemical compounds that are referred to as farnesene include both α and β isomers. The IUPAC name for α-farnesene is 3,7,11-trimethyldodeca-1,3,6,10-tetraene, its molecular mass is 204.36 g/mol and its molecular formula is C₁₅H₂₄. The IUPAC name for β-farnesene is 7,11-dimethyl-3-methylene-dodeca-1,6,10-triene. The structure of β-farnesene is represented by the following chemical formula (I):

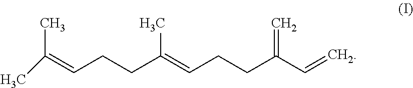

The unsaturated carboxylic acid is selected from unsaturated mono- and dicarboxylic acids, derivatives thereof, or mixtures thereof that can function as dienophiles in Diels-Alder reactions. “Derivatives” of carboxylic acids are defined herein as anhydrides, esters, amides, imides, aldehydes, ketones and nitriles. Suitable unsaturated carboxylic acids or derivatives for use in preparing the Diels-Alder adducts are maleic anhydride, itaconic anhydride, dimethyl maleate, dimethyl itaconate, maleic acid, itaconic acid, fumaric acid, dimethyl fumarate, acrylic acid, methyl acrylate, ethyl acrylate, butyl acrylate, methyl methacrylate, methacrylic acid, benzaldehyde, hydroxyethyl acrylate, hydroxyethyl methacrylate, hydroxypropyl acrylate, hydroxypropyl methacrylate, acrylonitrile, acrylamide, N-hydroxyethyl maleimide, maleimide, vinyl alkyl ketones (where the alkyl group may be methyl, ethyl, propyl, butyl, pentyl and hexyl), acrolein, methacrolein, and mixtures thereof. Particularly preferred are maleic anhydride, methyl acrylate, acrylonitrile, and itaconic anhydride.

When farnesene is reacted with maleic anhydride through a Diels-Alder reaction mechanism, a farnesene maleate adduct may be produced at a very fast rate (low cycle time and energy requirement) and high yield. A synthetic scheme for the production of a farnesene maleate adduct from a Diels-Alder reaction of β-farnesene (β-7,11-dimethyl-3-methylene-1,6,10-dodecatriene) with maleic anhydride is shown in Diels Alder Reaction Scheme A.

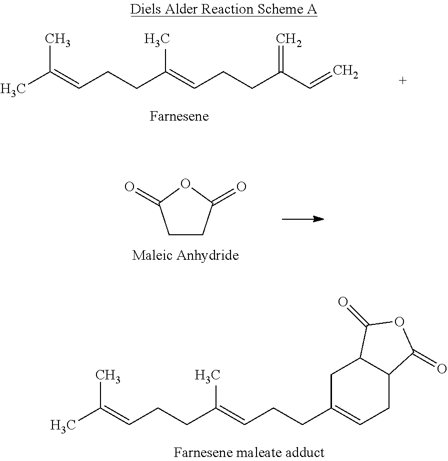

Representative schemes for preparing other Diels-Alder adducts for use in preparing the surfactants and solvents of the present technology are illustrated below. Note that in most cases, multiple isomers of the products are possible due to the nature of the Diels Alder reaction.

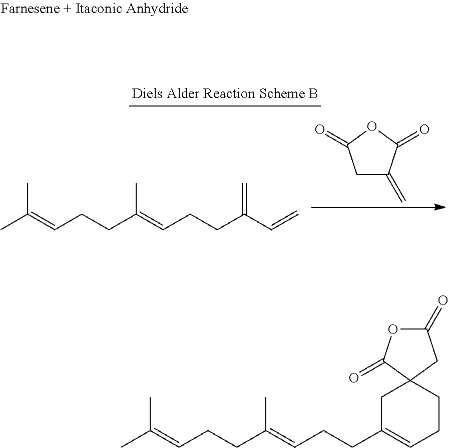

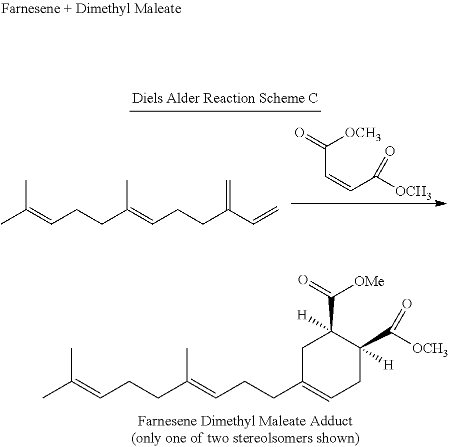

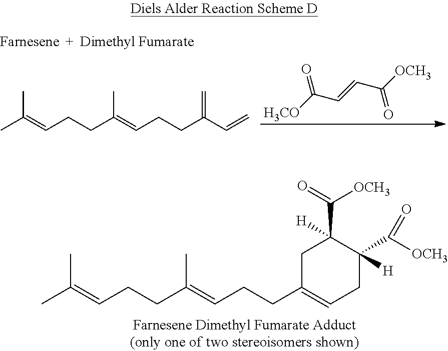

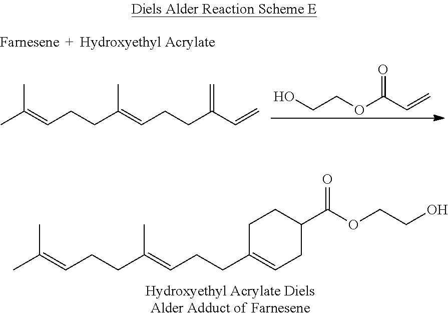

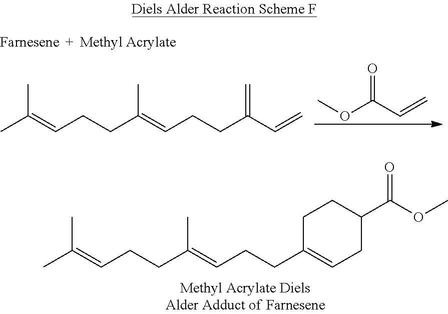

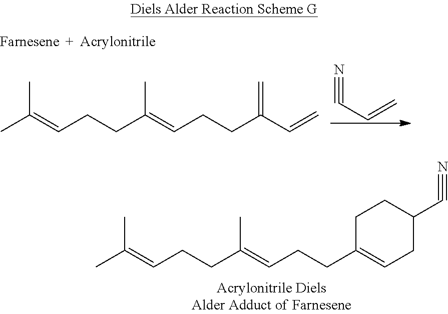

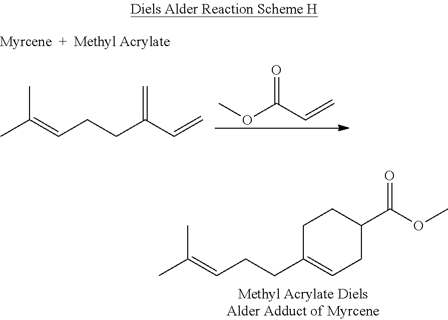

The reaction conditions for preparing the Diels-Alder adducts will depend upon the reactivity of the dienophile component with the biorenewable component. In general, the reaction temperature can vary over a wide range of temperatures of from about −50° C. to about 275° C. Solvents and catalysts can be used, if necessary, to facilitate the reaction of the components, although it is preferred that no solvents or catalysts be used since their removal potentially requires an additional processing step. The components are reacted for a sufficient time and under sufficient conditions to achieve at least a 60% conversion of the dienophile and biorenewable components into the Diels-Alder adduct.

### Derivatizing Adducts to Prepare Surfactants

The Diels-Alder adducts prepared by reacting the biorenewable material with the unsaturated carboxylic acid or derivative thereof are then derivatized to form the surfactants and solvents of the present technology. The derivatization procedures include (1) esterification with monoalkyl polyalkylene glycols (MPAGs) to form nonionic surfactants or solvents; (2) hydrogenation of an aryl-containing Diels Alder adduct of farnesene or myrcene to form a solvent, followed by sulfonation of the solvent to form an anionic surfactant; (3) amidation and/or imidation with a dialkylaminoalkyl amine followed by oxidation to form an amido and/or imido dialkyl amine oxide; (4) a ring opening reaction with one equivalent of mono-alkyl polyalkylene glycol (MPAG) followed by neutralization of the half acid with a base to form anionic surfactants; (5) hydrogenation of a nitrile-containing adduct, followed by reaction with methanol and subsequent quaternization with methyl or benzyl halide or dimethyl sulfate to form an ammonium halide or ammonium methyl sulfate; (6) ring opening or esterification with a sugar, optionally followed by neutralization to form a nonionic or anionic surfactant; (7) amidation with a mono- or di-alkyl amine to form an amide, followed by hydrogenation or reduction to form the dialkyl amine, followed by oxidation to form the dialkylamine oxide; (8) hydrogenation, followed by sulfonation, sulfation or phosphation; (9) hydrogenation of a nitrile containing adduct, followed by alkoxylation, followed by oxidation to form an alkoxylated amine oxide; (10) reaction with a mono- or oligo-ethyleneamine, or aminoethyl ethanolamine or mixtures thereof to form an imidazole, followed by quaternization with a suitable quaternizing agent such as an alkyl halide, an alkylaryl halide, epichlorohydrin or dimethyl sulfate to form quaternium surfactants; (11) alkoxylation with ethylene oxide, propylene oxide, or butylene oxide to form surfactants or solvents. In addition, (12) farnesene or myrcene may be reacted with a previously derivatized dienophile to form a surfactant or solvent. Finally, (13) farnesene or myrcene or mixtures thereof may be reacted directly with a sulfonating agent such as, for example, sodium or potassium bisulfite. Examples of several of these derivatization procedures are described in further detail below.

1. Esterification with MPAG

In one embodiment of the present technology, the Diels-Alder adducts containing carboxylic acids, esters or anhydrides are derivatized by esterifying the adducts with monoalkyl polyalkylene glycol (MPAG) to form nonionic surfactants. The monoalkyl group in the MPAG can have a carbon chain length of 1 to 6 carbons, and suitable MPAGs have a molecular weight in the range of about 100 to about 6,000, alternatively about 100 to about 4,000, preferably about 100 to about 1500. The MPAGs may be based on ethylene oxide, propylene oxide or butylene oxide, mixtures thereof or block copolymers thereof. In the case of carboxylic acid or anhydride based Diels-Alder adducts, water must be removed during the reaction to promote ester formation. In the case of ester based Diels-Alder adducts, alcohol must be removed during the transesterification to promote the formation of the desired ester. Solvents may be used to promote compatibilization of reagents and/or provide azeotropic removal of the water of reaction. Catalysis by acid or base may be used to promote the reaction. A representative reaction scheme for the esterification reaction is shown in Reaction Scheme 1:

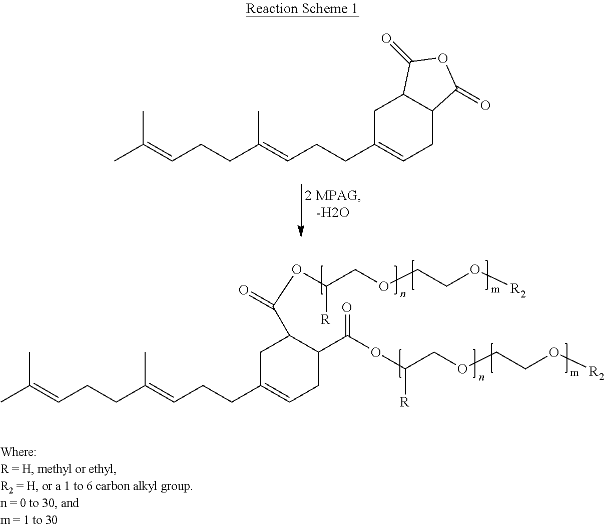

### 2. Hydrogenation and Sulfonation of Adducts

In another embodiment of the present technology, the Diels-Alder adducts containing carboxylic acids, esters or anhydrides are derivatized by esterifying the adducts with an aromatic-substituted hydroxy alkane, followed by hydrogenation. Suitable aromatic-substituted hydroxyl alkanes include 2-phenoxy ethanol, benzyl alcohol, 2-phenyl ethyl alcohol, and alkoxylated phenols having ethylene oxide and/or propylene oxide alkoxy groups. The aromatic-substituted hydroxy alkane ester adducts may be used “as is” in their hydrogenated or non-hydrogenated form as solvents. Alternatively, the hydrogenated adducts can be sulfonated with a sulfonation agent to form useful anionic surfactants. In the case of carboxylic acid or anhydride based Diels-Alder adducts, water must be removed during the reaction to promote ester formation. In the case of ester based Diels-Alder adducts, alcohol must be removed during the transesterification to promote the formation of the desired ester. Solvents may be used to promote compatibilization of reagents and/or provide azeotropic removal of the water of reaction. Catalysis by acid or base is preferred to promote the reaction. An exemplary reaction scheme for the esterification/hydrogenation/sulfonation reaction is shown in Reaction Scheme 2A and 2B:

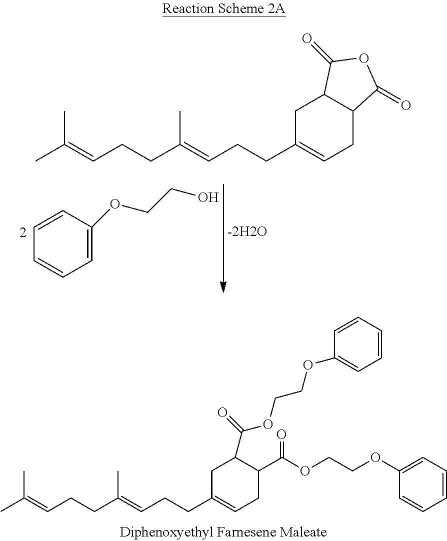

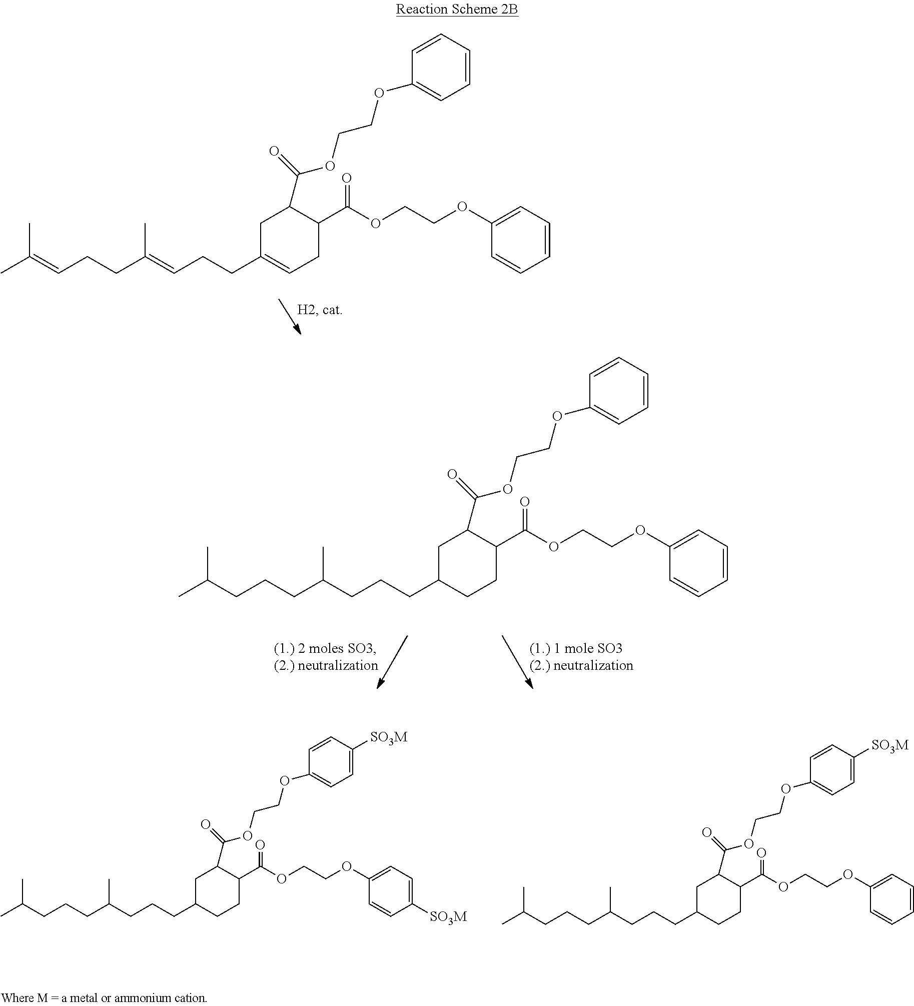

In another embodiment of the present technology, a dienophile containing an aldehyde and a phenyl or substituted phenyl ring may be reacted with either farnesene or myrcene to provide a Diels Alder adduct with useful solvent properties. This adduct may be hydrogenated to provide a solvent. This hydrogenated Diels Alder adduct may be sulfonated to provide products with useful surfactant properties. An example of this embodiment is represented in Reaction Scheme 2C.

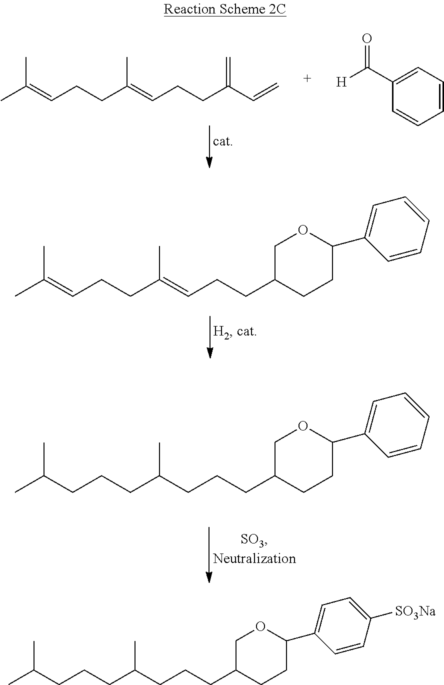

### 3. Amidation/Imidation Followed by Oxidation of Adducts

In another embodiment of the present technology, the Diels-Alder adducts are derivatized by amidation and/or imidation with a dialkyl alkylamine, followed by oxidation. Diels Alder adducts containing carboxylic acids, esters or anhydrides may be amidated and/or imidated with dialkylamino alkylamines. Contemplated dialkylamino alkylamines are those having 1 to about 4 carbons in the N,N alkyl moiety and 1 to about 6 carbon atoms in the alkylamine moiety. Suitable dialkylamino alkylamines include N,N-dimethyl amino propyl amine, N,N-dimethyl amino ethyl amine, N,N-diethyl amino propylamine, and N,N-dibutyl amino propyl amine. In the case of carboxylic acids or anhydrides, water must be removed during the reaction to promote amide/imide formation. In the case of esters, alcohol must be removed during the amidation/imidation to promote the formation of the desired amide and/or imide. Solvents may be used to promote compatibilization of reagents and/or provide azeotropic removal of the water of reaction. Contemplated solvents include toluene, xylene and dichlorobenzene. A large excess of dialkylamino alkylamine may be used to promote the reaction followed by removal of the excess by distillation. The formation of the dialkyl amino alkyl amide and/or imide derivative is followed by an oxidation step to form the dialkyl amine oxide derivative. Contemplated oxidizing agents are hydrogen peroxide and peracetic acid. Representative schemes for the amidation and/or imidation followed by oxidation reaction are shown in reaction schemes 3A and 3B:

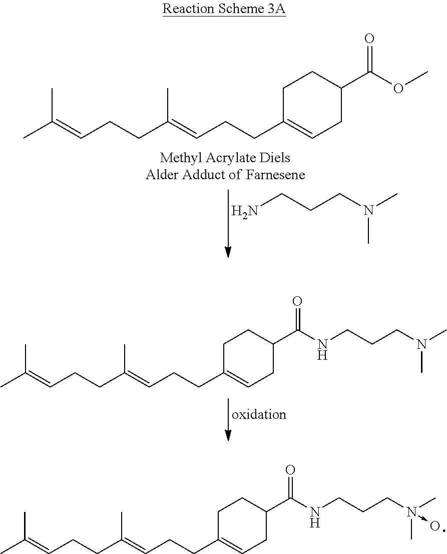

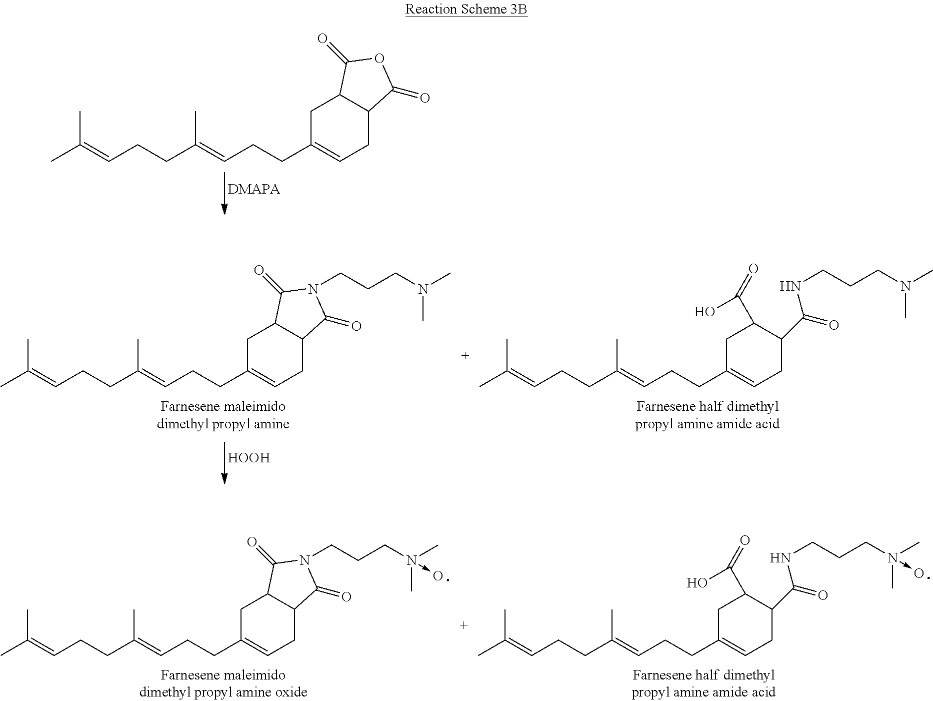

Note that depending on the conversion to imide, the final product may be the pure imide, a mixture of imide with amide, or the pure amide. The amine oxide derivatives described above may be used in surfactant applications, while the amine derivatives may be utilized in solvent applications.

### 4. Ring Opening/Neutralization of Adduct

In another embodiment of the present technology, the anhydride-containing Diels-Alder adducts are derivatized by ring opening the adduct with one equivalent of mono-alkyl ether of polyalkylene glycol, followed by neutralization with a base. The anhydride ring may be opened by MPAG by simply mixing equimolar amounts of each together with exposure to mild heating (room temperature to about 130° C.). No catalyst is needed to form the half-acid ester. MPAGs having between 1 and 30 moles of ethylene oxide are useful for preparing these adducts. The resulting acid is preferably neutralized to a pH between 4.5 and 8.5 using a suitable base, such as, for example sodium hydroxide, potassium hydroxide, triethanol amine, triethyl amine, methyl diethanol amine, sodium carbonate, and the like. The neutralization step may occur at temperatures between 5° C. and 95° C. Water may optionally be present to facilitate the neutralization step, and may be concurrently added with the base. An example of the ring opening/neutralization reaction is shown in Reaction Scheme 4:

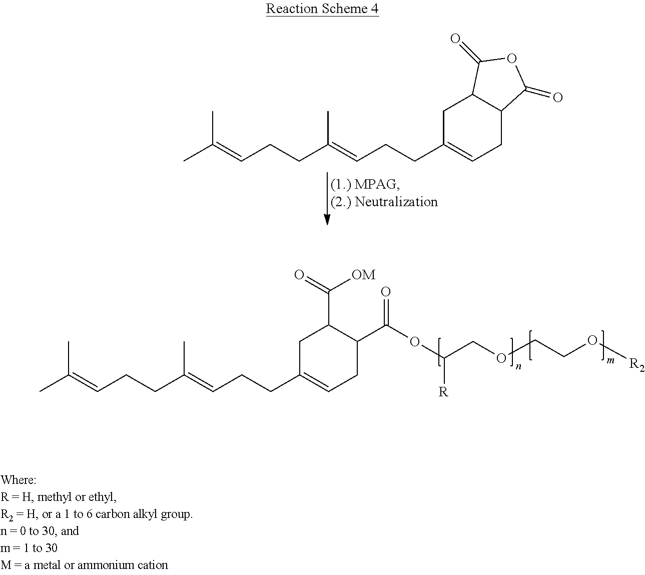

These derivatives are useful as anionic surfactants.

### 5. Hydrogenation/Quaternization of Adduct

In another embodiment of the present technology, a Diels-Alder adduct formed from the reaction of farnesene or myrcene with a nitrile such as acrylonitrile or methacrylonitrile, for example, is derivatized by hydrogenation to form an amine. The amine is then reacted with, for example, methanol to form a tertiary amine, followed by quaternization by reaction of the amine with methyl chloride, benzyl chloride, or dimethyl sulfate. An example of the reaction scheme for the hydrogenation/quaternization reaction is shown below in Reaction Scheme 5A:

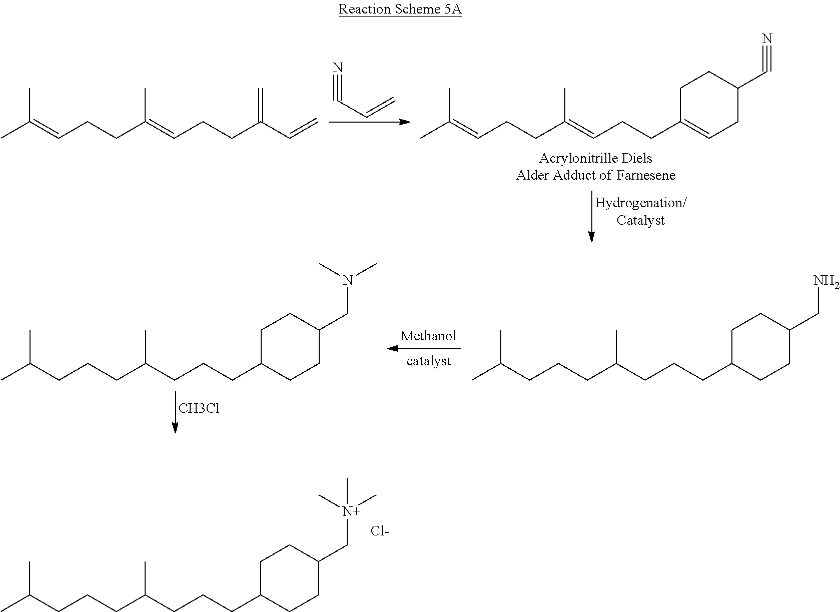

An example of a process suitable for hydrogenating the nitrile derivative to form a primary amine may be found in U.S. Pat. No. 5,175,370. Industrially, the alkylation of primary amines is typically conducted using alcohols, not alkyl halides, as shown above. Alcohols are less expensive than alkyl halides and their alkylation does not produce salts, the disposal of which is problematic. The key to the alkylation of alcohols is the use of catalysts that render the hydroxyl group a good leaving group. Many industrially significant tertiary alkylamines are produced from primary amines and alcohols such as methanol. (Karsten Eller, Erhard Henkes, Roland Rossbacher, Hartmut Höke “Amines, Aliphatic” in Ullmann's Encyclopedia of Industrial Chemistry, Wiley-VCH, Weinheim, 2005.) The resulting tertiary amine may then be conveniently quaternized using a quaternizing reagent such as methyl chloride, methyl sulfate, or benzyl chloride. The resulting tertiary amine may also be oxidized using, for example, hydrogen peroxide, to provide an amine oxide. The cationic surfactants may be used in such applications as corrosion resistance compositions, biocidal compositions, and hair conditioners, for example. The amine oxide surfactants may be used in light duty liquid detergents, hard surface cleaning formulations, and agricultural adjuvant applications, for example. In a further embodiment of the present technology, sulfobetaines may be also be prepared by reacting amine-containing Diels Alder adduct derivatives of farnesene, myrcene or mixtures thereof with epichlorohydrin and a bisulfite, as shown in the example provided in Reaction Scheme 5B, below.

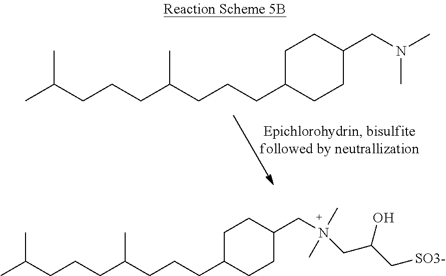

6. Esterification with a Sugar

In another embodiment of the present technology, the carboxylic acid, anhydride or ester containing Diels-Alder adducts are derivatized by esterifying the adduct with a sugar. Suitable sugars include glycerol, sucrose, glucose, dextrose, sorbitol, polyglycerol, fructose, lactose and sugars derived from the chemical of enzymatic treatment of biorenewable cellulose. Representative schemes for the esterification reaction are shown in Reaction Schemes 6A and 6B:

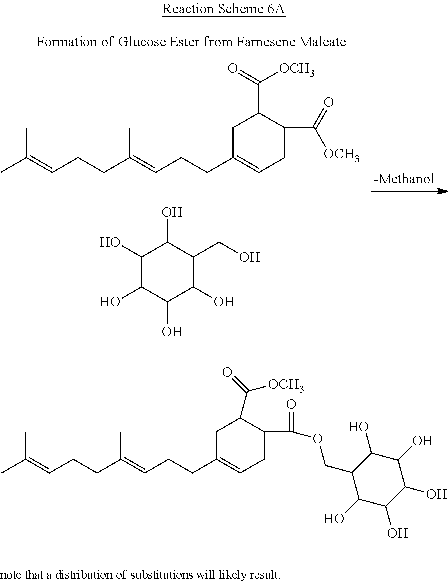 [SMILES: COC(=O)C1CC=C(CC/C=C(\C)CCC=C(C)C)CC1C(=O)OC ; OCC1C(O)C(O)C(O)C(O)C1O ; COC(=O)C1CC(CC/C=C(\C)CCC=C(C)C)=CCC1C(=O)OCC1C(O)C(O)C(O)C(O)C1O]

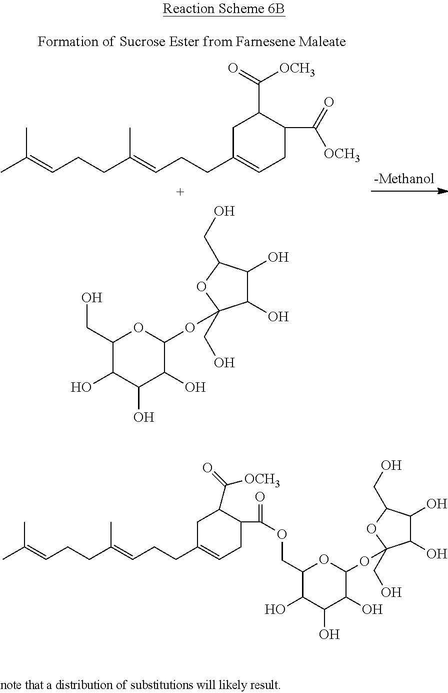 [SMILES: COC(=O)C1CC=C(CC/C=C(\C)CCC=C(C)C)CC1C(=O)OC ; OCC1OC(OC2(CO)OC(CO)C(O)C2O)C(O)C(O)C1O ; COC(=O)C1CC(CC/C=C(\C)CCC=C(C)C)=CCC1C(=O)OCC1OC(OC2(CO)OC(CO)C(O)C2O)C(O)C(O)C1O]

These derivatives are expected to have useful surfactant properties.

7. Amidation with an Alkyl Amine or Dialkyl Amine

In another embodiment of the present technology, the Diels-Alder adducts containing esters, anhydrides or carboxylic acids are derivatized by amidation of the adducts with an alkyl amine or dialkyl amine. Contemplated alkyl amines or dialkyl amines are those having 1 to about 4 carbons in the alkyl or dialkyl moiety. Suitable alkyl or dialkyl amines include methyl amine, ethyl amine, dimethyl amine, diethyl amine and dibutyl amine. A reaction scheme for the amidation reaction is shown in Reaction Scheme 7A, below. These amide derivatives can be used as solvents and in cleaning applications.

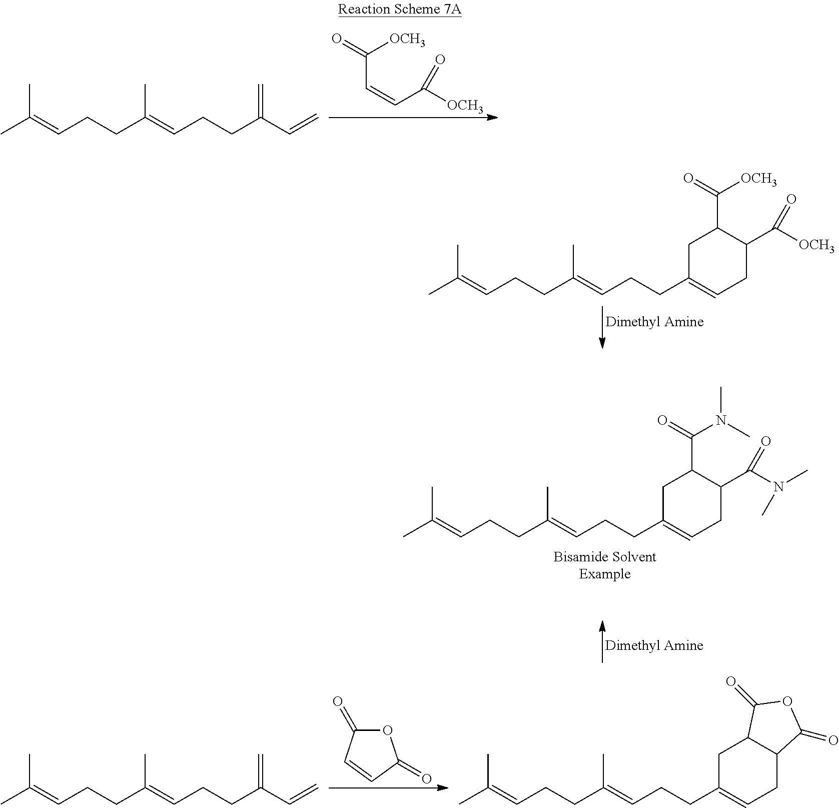

Additionally, amides from this process may be hydrogenated and subsequently oxidized with, for example, hydrogen peroxide to provide useful amine oxide surfactants as shown in Scheme 7B.

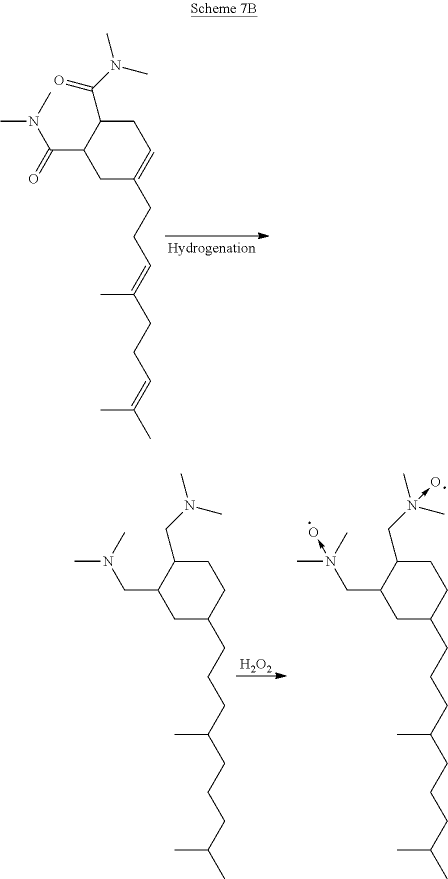

### 8. Hydrogenation Followed by Sulfation or Phosphation

In another embodiment of the present technology, Diels Alder adducts containing ketones, hydroxyls, aldehydes, carboxylic acids, esters and anhydrides may be hydrogenated to form alcohols followed by either sulfation or phosphation of the resulting product. The alcohols may be optionally alkoxylated using ethylene oxide, propylene oxide, butylene oxide or mixtures thereof prior to sulfation. Examples of this embodiment are represented by Reaction Schemes 8A through 8F.

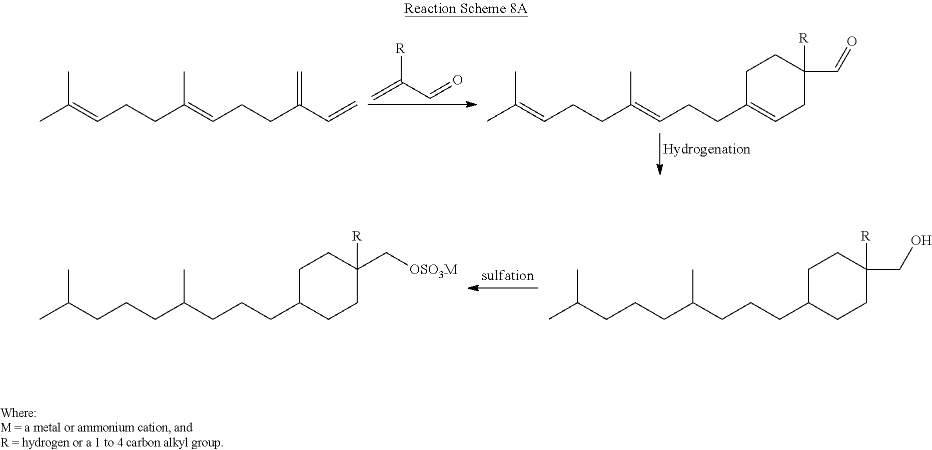

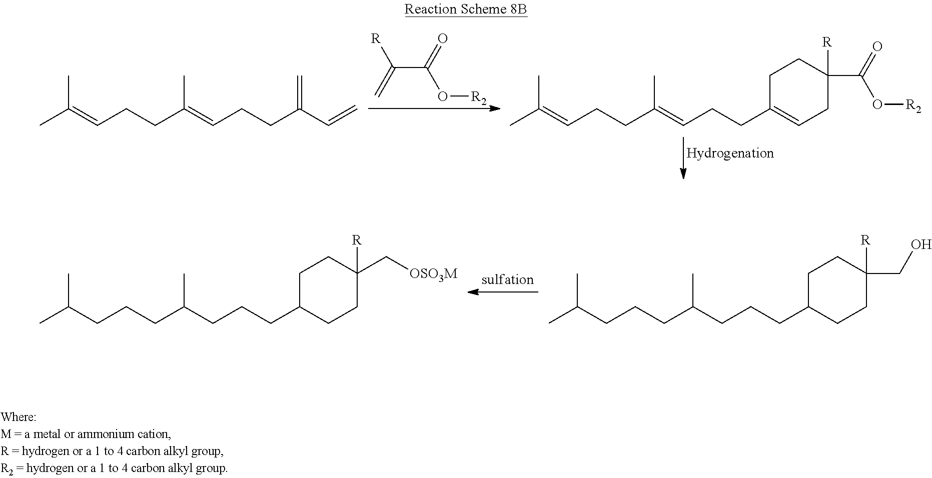

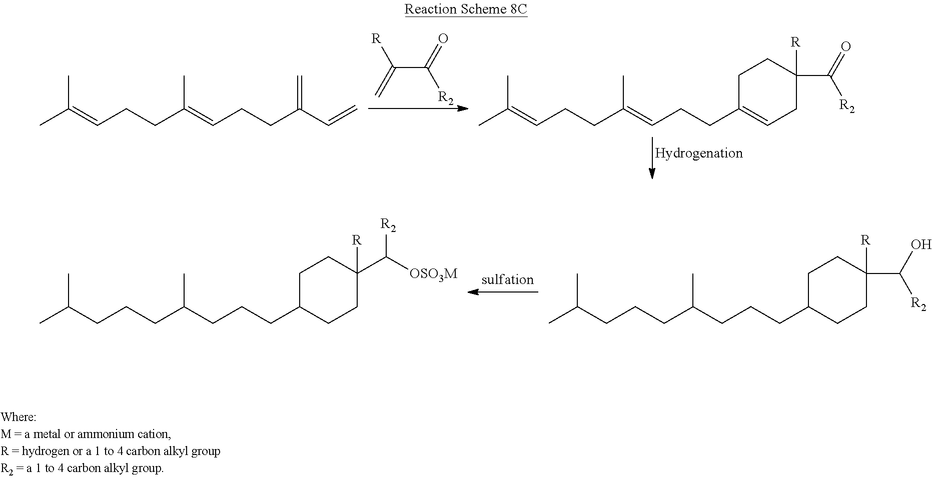

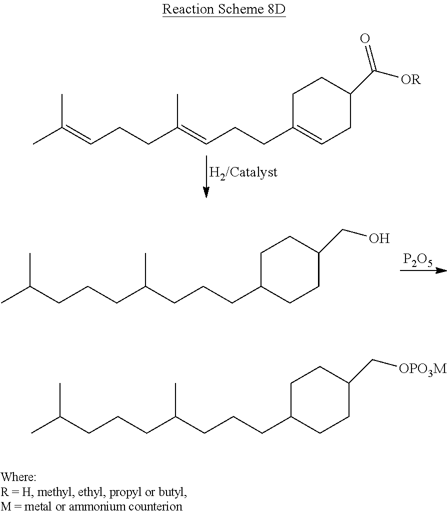

The alcohols may also be reacted with cyclic sultones such as 1,3-propane sultone, 1,4-propane sultone, or their alkyl derivatives to provide sulfonates, as indicated by the example shown in Reaction Scheme 8E, below.

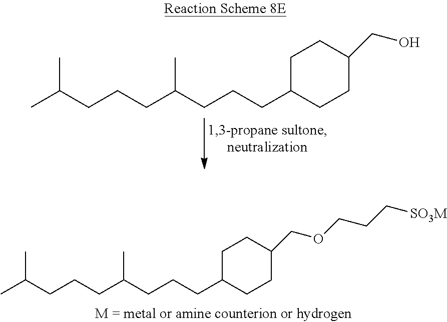

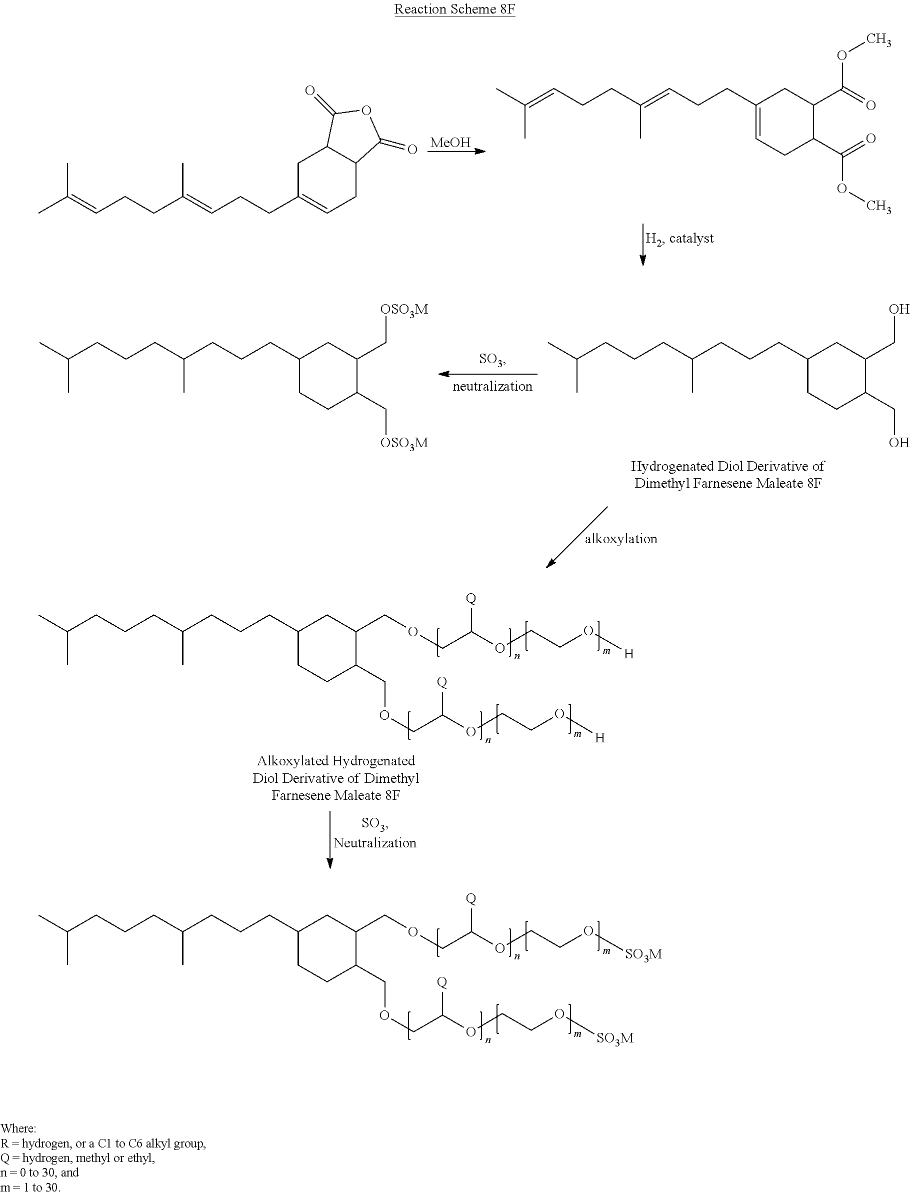

### 9. Hydrogenation of a Nitrile Adduct, Followed by Alkoxylation, Followed by Oxidation

In another embodiment of the present technology, a nitrile-containing Diels Alder adduct of farnesene or myrcene may be hydrogenated, then alkoxylated and then oxidized to form an alkoxylated amine oxide. An example of this embodiment is represented by Reaction Scheme 9.

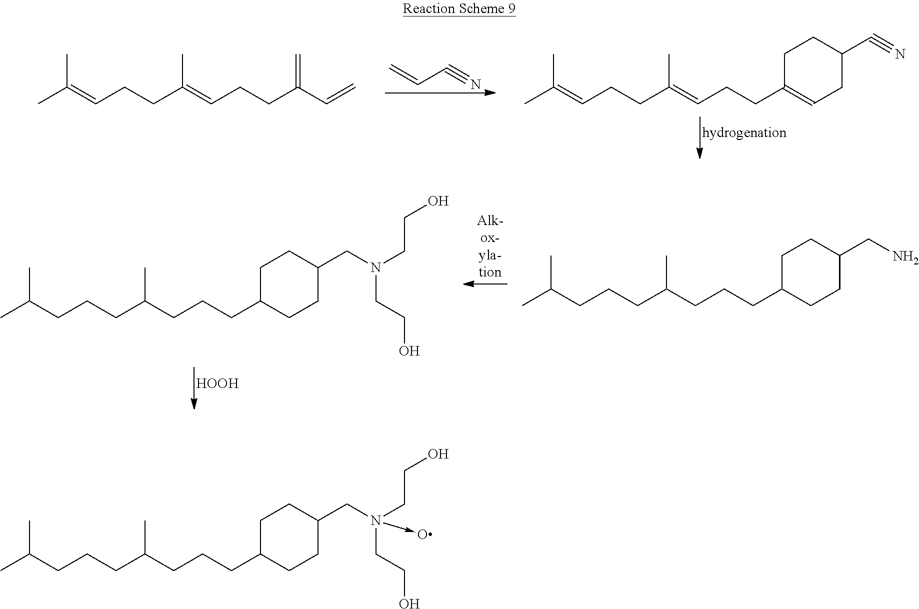

### 10. Imidazoline Formation Followed by Quaternization

In another embodiment of the present technology, an ester-containing Diels Alder derivative of farnesene or myrcene may be reacted with mono- or oligo-ethyleneamine, or aminoethyl ethanolamine or mixtures thereof to form an imidazole, followed by quaternization with a suitable quaternizing agent such as an alkyl halide, an alkylaryl halide or dimethyl sulfate. An example of this embodiment is represented by Reaction Scheme 10.

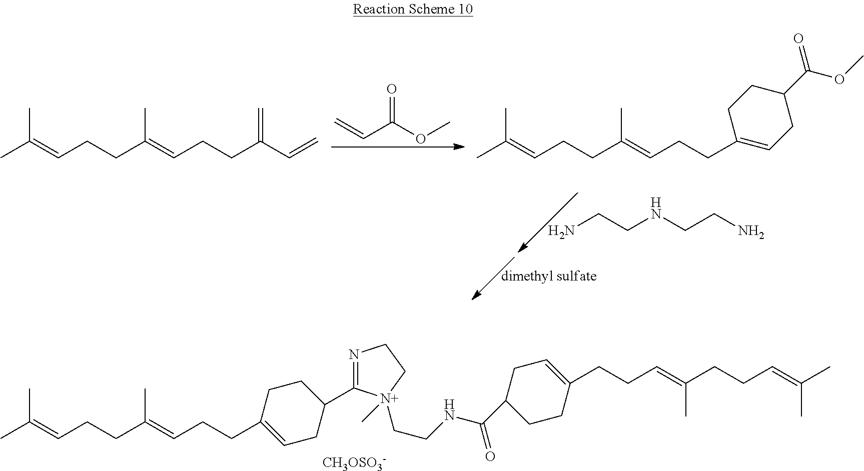

### 11. Hydrogenation Followed by Alkoxylation

In another embodiment of the present technology, Diels Alder adducts containing ketones, hydroxyls, aldehydes, carboxylic acids, esters and anhydrides may be hydrogenated to form alcohols, which may then be alkoxylated with ethylene oxide, propylene oxide, butylene oxide or mixtures or block copolymers thereof to provide nonionic surfactants and solvents. An example of this embodiment is represented by Reaction Scheme 11.

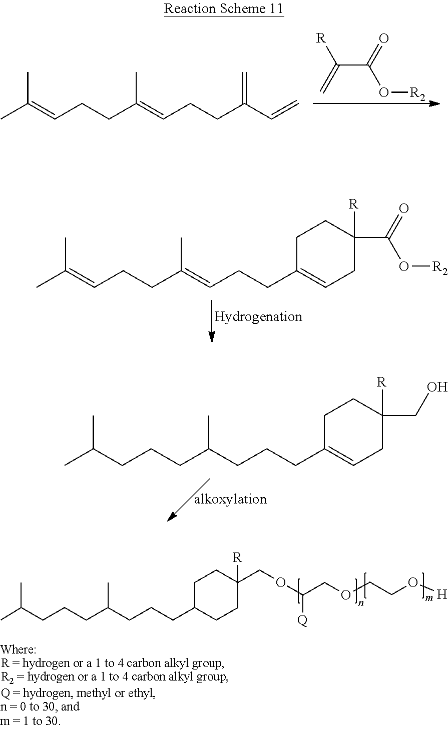

12. Diels Alder Reaction of Farnesene or Myrcene with a Derivatized Dienophile

In another embodiment of the present technology, a dienophile containing or derivatized using a mono-alkyl ether of polyalkylene glycol, a mono-alkyl ether of polypropyloxy-polyethyloxy block copolymer, an amine-containing mono-alkylene glycol, an amine-containing polyalkylene glycol, a mono-alkyl ether of polybutyloxy-polyethyloxy block copolymer, a polyalkylene glycol containing ethylene oxide, propylene oxide, butylene oxide or mixtures thereof, an alkylene glycol, an amine, or a polyamine, or mixtures thereof may be reacted with either farnesene or myrcene to provide a Diels Alder adduct with useful surfactant or solvent properties or both. An example of this embodiment is provided in Reaction Schemes 12A and 12B.

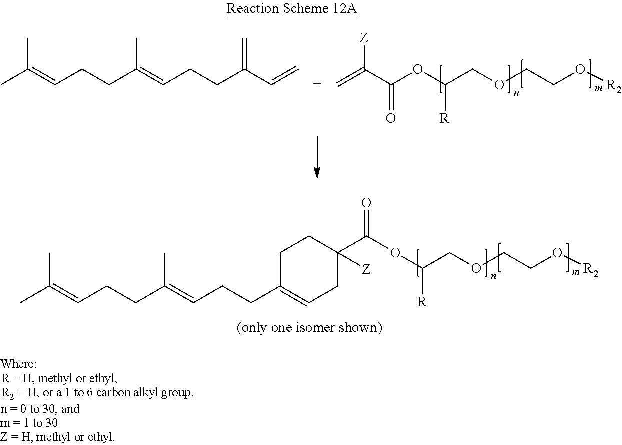

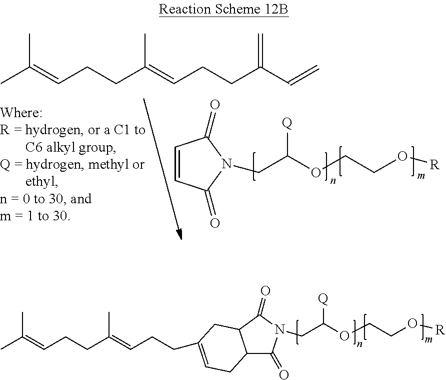

### 13. Direct Sulfonation of Farnesene

In another embodiment of the present technology, farnesene, myrcene and mixtures thereof may be sulfonated using sodium or potassium bisulfite (Reaction Scheme 13, below). An example procedure for the sulfonation of alpha-olefins is provided in U.S. Pat. No. 3,622,517.

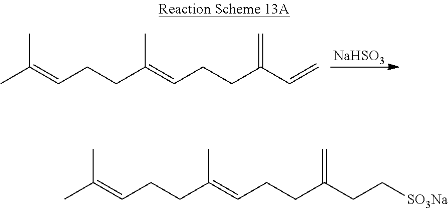

In another embodiment of the present technology, farnesene, myrcene and mixtures thereof may be sulfonated using sulfur dioxide, sodium sulfite and a free radical initiator such as, for example, t-peroxy benzoate (Reaction Scheme 13B, below). An example procedure for this route is described in R. Herke, K. Rasheed, JAOCS, vol. 69, no. 1, (January, 1992), pg. 47-51.

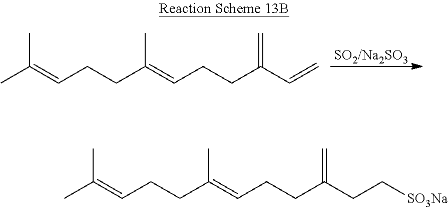

The surfactants and solvents of the present technology can be used in a wide variety of applications. For example, some applications for the surfactants and solvents of the present technology include personal care products, such as shampoos, body washes and liquid or solid soaps; cleaning compositions, such as liquid hand dishwashing compositions, machine dishwashing compositions, and hard surface cleaners; laundry detergents; fabric softeners; agricultural compositions; and oil field and oil recovery applications.

Compositions that include the surfactants or solvents of the present technology will also typically include one or more co-surfactants selected from anionic, nonionic, cationic, ampholytic and zwitterionic surfactants or mixtures thereof. Suitable anionic surfactants for use as a co-surfactant include carboxylic acid salts, represented by the formula:

R¹COOM

where R¹ is a primary or secondary alkyl group of 4 to 30 carbon atoms and M is a solubilizing cation. The alkyl group represented by R¹ may represent a mixture of chain lengths and may be saturated or unsaturated, although it is preferred that at least two thirds of the R¹ groups have a chain length of between 8 and 18 carbon atoms. Non-limiting examples of suitable alkyl group sources include the fatty acids derived from coconut oil, tallow, tall oil and palm kernel oil. The solubilizing cation, M, may be any cation that confers water solubility to the product, although monovalent moieties are generally preferred. Examples of acceptable solubilizing cations for use with the present technology include alkali metals such as sodium and potassium, and amines such as triethanolammonium, ammonium and morpholinium.

Primary alkyl sulfates are represented by the formula:

R²OSO₃M

where R² is a primary alkyl group of 8 to 18 carbon atoms. M is H or a cation, e.g., an alkali metal cation (e.g. sodium, potassium, lithium), or ammonium or substituted ammonium (e.g. methyl-, dimethyl-, and trimethyl ammonium cations and quaternary ammonium cations such as tetramethyl-ammonium and dimethyl piperidinium cations and quaternary ammonium cations derived from alkylamines such as ethylamine, diethylamine, triethylamine, and mixtures thereof). The alkyl group R² may have a mixture of chain lengths. It is preferred that at least two-thirds of the R² alkyl groups have a chain length of 8 to 14 carbon atoms. This will be the case if R² is coconut alkyl, for example. The solubilizing cation may be a range of cations which are in general monovalent and confer water solubility, such as, for example alkali metal cations. Other possibilities are ammonium and substituted ammonium ions, such as trialkanolammonium or trialkylammonium.

Other suitable anionic surfactants that can be used are alkyl ester sulfonate surfactants including linear esters of C₈-C₂₀ carboxylic acids (i.e., fatty acids) which are sulfonated with gaseous SO₃ according to “The Journal of the American Oil Chemists Society”, 52 (1975), pp. 323-329. Suitable starting materials would include natural fatty substances as derived from tallow, palm oil, etc.

Alkyl benzene sulfonates are represented by the formula:

R⁶ArSO₃M

where R⁶ is an alkyl group of 8 to 18 carbon atoms, Ar is a benzene ring (—C₆H₄—) and M is a solubilizing cation. The group R⁶ may be a mixture of chain lengths. A mixture of isomers is typically used, and a number of different grades, such as “high 2-phenyl” and “low 2-phenyl” are commercially available for use depending on formulation needs.

Paraffin sulfonates having about 8 to about 22 carbon atoms, preferably about 12 to about 16 carbon atoms, in the alkyl moiety, and olefin sulfonates having 8 to 22 carbon atoms, preferably 12 to 16 carbon atoms, are also contemplated anionic surfactants for use herein.

Sulfosuccinate esters represented by the formula:

R⁷OOCCH₂CH(SO₃⁻M⁺)COOR⁸

are also useful in the context of the present technology. R⁷ and R⁸ are alkyl groups with chain lengths of between 2 and 16 carbons, and may be linear or branched, saturated or unsaturated.

Organic phosphate based anionic surfactants include organic phosphate esters such as complex mono- or diester phosphates of hydroxyl-terminated alkoxide condensates, or salts thereof. Included in the organic phosphate esters are phosphate ester derivatives of polyoxyalkylated alkylaryl phosphate esters, of ethoxylated linear alcohols and ethoxylates of phenol. Also included are nonionic alkoxylates having a sodium alkylenecarboxylate moiety linked to a terminal hydroxyl group of the nonionic through an ether bond. Counterions to the salts of all the foregoing may be those of alkali metal, alkaline earth metal, ammonium, alkanolammonium and alkyl ammonium types.

### Nonionic Surfactants

Suitable nonionic surfactants for use as a co-surfactant in the present compositions include alkyl polyglucosides (“APGs”), alcohol ethoxylates, nonylphenol ethoxylates, and others.

Other suitable nonionic surfactants are poly hydroxy fatty acid amide surfactants of the formula:

R²—C(O)—N(R¹)—Z

where R¹ is H, or R¹ is C₁₋₄ hydrocarbyl, 2-hydroxyethyl, 2-hydroxypropyl or a mixture thereof, R² is C₅₋₃₁ hydrocarbyl, and Z is a polyhydroxy hydrocarbyl having a linear hydrocarbyl chain with at least 3 hydroxyls directly connected to the chain, or an alkoxylated derivative thereof. Preferably, R¹ is methyl, R² is a straight C₁₁₋₁₅ alkyl or alkenyl chain such as coconut alkyl or mixtures thereof, and Z is derived from a reducing sugar such as glucose, fructose, maltose, lactose, in a reductive amination reaction.

Other suitable nonionics are amine oxide surfactants. The compositions of the present technology may comprise amine oxide in accordance with the general formula:

R¹(EO)ₓ(PO)_{y}(BO)_{z}N(O)(CH₂R′)₂.H₂O

In general, it can be seen that the preceding formula provides one long-chain moiety R¹(EO)ₓ(PO)_{y}(BO)_{z} and two short chain moieties, —CH₂R′. R′ is preferably selected from hydrogen, methyl and —CH₂OH. In general R¹ is a primary or branched hydrocarbyl moiety which can be saturated or unsaturated, preferably, R¹ is a primary alkyl moiety. When x+y+z=0, R¹ is a hydrocarbyl moiety having a chain length of from about 8 to about 18. When x+y+z is different from 0, R¹ may be somewhat longer, having a chain length in the range C₁₂-C₂₄. The general formula also encompasses amine oxides where x+y+z=0, R¹ is C₈-C₁₈, R′ is H and q=from 0 to 2, preferably 2. These amine oxides are illustrated by C₁₂₋₁₄ alkyldimethyl amine oxide, hexadecyl dimethylamine oxide, octadcylamine oxide and their hydrates, especially the dihydrates as disclosed in U.S. Pat. Nos. 5,075,501 and 5,071,594, which are incorporated herein by reference.

The presently described technology also encompasses amine oxides where x+y+z is different from zero, specifically x+y+z is from about 1 to about 10, and R¹ is a primary alkyl group containing about 8 to about 24 carbons, preferably from about 12 to about 16 carbon atoms. In these embodiments y+z is preferably 0 and x is preferably from about 1 to about 6, more preferably from about 2 to about 4; EO represents ethyleneoxy; PO represents propyleneoxy; and BO represents butyleneoxy. Such amine oxides can be prepared by conventional synthetic methods, e.g., by the reaction of alkylethoxysulfates with dimethylamine followed by oxidation of the ethoxylated amine with hydrogen peroxide.

### Cationic Surfactants

Cationic surfactants suitable for use as co-surfactants include ditallow dimethylammonium chloride (DTDMAC), fatty alkanolamides (FAA), and quaternized diesters of trialkanolamines and fatty acids.

Cationic detersive surfactants suitable for use in the compositions of the present technology include those having one long-chain hydrocarbyl group. Examples of such cationic surfactants include the ammonium surfactants such as alkyldimethylammonium halogenides, and those surfactants having the formula:

[R²(OR³)_{y}][R⁴(OR³)_{y}]₂R⁵N⁺X⁻

where R² is an alkyl or alkyl benzyl group having from about 8 to about 18 carbon atoms in the alkyl chain, each R³ is selected from the group consisting of —CH₂CH₂—, —CH₂CH(CH₃)—, —CH₂CH(CH₂OH)—, —CH₂CH₂CH₂—, and mixtures thereof; each R⁴ is selected from the group consisting of C₁-C₄ alkyl, C₁-C₄ hydroxyalkyl, benzyl ring structures formed by joining the two R⁴ groups, —CH₂CHOH—CH(OH)C(O)R⁶CH(OH)CH₂OH where R⁶ is any hexose or hexose polymer having a molecular weight less than about 1000, and hydrogen when y is not 0; R⁵ is the same as R⁴ or is an alkyl chain where the total number of carbon atoms of R² plus R⁵ is not more than about 18; each y is from 0 to about 10 and the sum of the y values is from 0 to about 15; and X is any compatible anion. The long chain cationic surfactant can also be the quaternized version of stearamidopropyl dimethylamine (e.g. stearamidopropyl trimethylamine chloride).

Other suitable cationic surfactants are the water-soluble quaternary ammonium compounds having the formula:

R¹R²R³R⁴N⁺X⁻

where R¹ is C₈-C₁₆ alkyl, each of R², R³ and R⁴ is independently C₁-C₄ alkyl, C₁-C₄ hydroxyalkyl, benzyl, or —(C₂H₄O)ₓ H where x has a value from 1 to 5, and X is an anion. In an embodiment, not more than one of R², R³ or R⁴ is benzyl.

### Ampholytic Surfactants

Ampholytic surfactants can be broadly described as aliphatic derivatives of heterocyclic secondary and tertiary amines, in which the aliphatic radical may be straight chain or branched and where one of the aliphatic substituents contains from about 8 to about 18 carbon atoms and at least one contains an anionic water-solubilizing group, e.g., carboxy, sulfo, sulfato, phosphato or phosphono (see U.S. Pat. No. 3,664,961, which provides specific examples of ampholytic surfactants from col. 6, line 60, to col. 7, line 53, incorporated here by reference). Examples of suitable ampholytic surfactants include fatty amine oxides and fatty amidopropylamine oxides. A specific suitable example is cocoamidopropyl betaine (CAPB) also known as coco betaine.

### Zwitterionic Surfactants

Zwitterionic synthetic detergents can be broadly described as derivatives of aliphatic quaternary ammonium and phosphonium or tertiary sulfonium compounds, in which the cationic atom may be part of a heterocyclic ring, and in which the aliphatic radical may be straight chain or branched, and where one of the aliphatic substituents contains from about 3 to 18 carbon atoms, and at least one aliphatic substituent contains an anionic water-solubilizing group, e.g., carboxy, sulfo, sulfato, phosphato or phosphono. (see U.S. Pat. No. 3,664,961, which provides specific examples of zwitterionic surfactants from col. 7, line 65, to col. 8, line 75, incorporated here by reference).

The compositions of the present technology also typically include one or more adjuncts, such as, for example, builders, enzymes, soil suspending agents, soil release and antiredeposition agents, chelating agents, dispersing agents, stabilizers, pH control agents, colorants, brighteners, dyes, anti-fading agents, whiteness enhancers, color maintenance agents, color restoration agents, dye fixatives, dye transfer agents, odor control agents, perfumes, pro-perfumes, cyclodextrin, preservatives, anti-oxidants, anti-corrosion agents, sanitation agents, antimicrobial agents, disinfecting agents, pesticides, anti-abrasion agents, rinse aids, flame retardants, water proofing agents, suds suppressors, fabric treatment agents and UV protection agents.

### EXAMPLES

The following abbreviations may be used in the present application, especially in the following Examples:

Ammonyx LO: Lauramine oxide
Ammonyx LMDO: Lauramidopropylamine/Myristamidopropylamine oxide

### CMC Critical Micelle Concentration

DiMTEG: Dimethyl triethylene glycol
DMAPA: Dimethyl amidopropyl amine
MPEG-12: Monomethyl ether of polyethylene glycol having an average of 12 moles of ethoxylation

### NaLAS: Sodium Lauryl Alcohol Sulfate

MPEG: Mono-methyl ether of polyethylene glycol

The following test methods are used in the examples of the present application.

### Shake Foam Test Method

This method determines the foam height and foam stability properties of surface active agents. A dilute surfactant solution and castor oil, when it is used, are added to a graduated cylinder. (It should be noted that in some experiments in the examples, castor oil is utilized and in other experiments, it is not utilized.) The shake foam machine inverts the cylinder for a specified number of inversions. The foam is allowed to settle for a brief time, and the foam height is recorded. After 5 minutes has elapsed, the foam height is measured again.

Apparatus:
1. Shake foam machine
2. Cylinder, graduated, 500 mL, with rubber stopper; and (3) analytical balance, +/−0.01 g.

Reagents:
1. Tap water (however, it should be understood by those skilled in the art that other types of water, such as deionized water or water with higher and lower water hardness, can also be used in the practice of the present technology), at 25° C., and
2. Castor oil.

Procedure:
1. A 0.2% active sample solution is prepared in 25° C. tap water. A 0.2% solids solution is prepared if the active level is unknown.
2. 100.0 g+/−0.01 g, of the 0.2% sample solution is added to a 500 mL graduated cylinder. The initial foam is kept to a minimum.
3. 2.0 g+/−0.01 g of castor oil is added to the graduated cylinder, and a stopper is placed on the cylinder.
4. The graduated cylinder is placed in the shake foam machine, and the clamps are secured at the rubber stopper.
5. The shake foam machine is programmed to invert cylinder 10 times.
6. The foam is allowed to settle for 15 seconds. A reading of total foam height, including the base of the 100 mL solution, is taken.
7. After 5 minutes, foam height is read and recorded again as in Step 6.

### Draves Wetting Test Method

The Draves Wetting test method is based on ASTM procedure D2281 and determines the wetting efficiency of a wetting agent. In accordance with the test procedure, a weighted cotton test skein is dropped into a tall cylinder containing a wetting agent having a 0.1% actives concentration dissolved in water. The time required (in seconds) for the cotton skein to wet through and sink, relaxing the string stirrup to which it is attached is recorded as the sinking time. This time relates to the speed at which the wetting agent works and can be used to compare agents.

### Gardner Cleaning Test for Hard-Surface Cleaners

This test measures the ability of a cleaning product to remove a greasy dirt soil from a white vinyl tile. The test is automated and uses an industry standard Gardner Straight Line Washability Apparatus. A camera and controlled lighting are used to take a live video of the cleaning process. The machine uses a sponge wetted with a known amount of test product. As the machine wipes the sponge across the soiled tile, the video records the result, from which a cleaning percentage can be determined. A total of 10 strokes are made, and cleaning is calculated for each of strokes 1-10 to provide a profile of the cleaning efficiency of the product.

### Example 1

### Farnesene/Itaconic Anhydride Diels-Alder Adduct

Itaconic anhydride (56 g/0.50 mole) and beta-farnesene (104 g/0.51 mole) were added to a 250 mL, three-neck, round bottom flask equipped with a heating mantle, nitrogen purge, magnetic stirring, thermocouple and condenser. The mixture was heated to 80° C., when the appearance of the mixture changed from opalescent to transparent and an exotherm resulted in a temperature spike to 243° C. The heating mantle was removed and the stirred mixture allowed to cool to room temperature. Gel phase permeation chromatography indicated that the product is the Diels-Alder adduct of farnesene and itaconic anhydride.

### Example 2

### Myrcene/Maleic Anhydride Diels-Alder Adduct

Maleic Anhydride (67.6 g/0.69 mole) and beta-myrcene (95.6 g/0.70 mole) were added to a 250 mL, three-neck, round bottom flask equipped with a heating mantle, magnetic stirring, thermocouple nitrogen purge and condenser. The mixture was heated to 50° C., when the appearance of the mixture changed from opalescent to transparent and an exotherm resulted in a temperature spike to about 230° C. Vigorous reflux occurred. The heating mantle was removed and the stirred mixture allowed to cool to room temperature. Gel phase permeation chromatography indicated that the product is the Diels-Alder adduct of myrcene and maleic anhydride.

### Example 3

### Farnesene/Hydroxyethyl Acrylate Diels-Alder Adduct

| Component | Wt., g | mw | moles |
|---|---|---|---|
| Farnesene | 61.59 | 204.35 | 0.30 |
| Hydroxyethyl acrylate | 35.00 | 116.12 | 0.30 |
| Wt. total = | 96.59 | | |

Hydroxyethyl acrylate was added dropwise to beta-farnesene over a period of about one hour at 50° C. with no N2 purge. The exotherm was minimal, so the temperature was raised over an hour to 180° C. for 6 hours to determine if the reaction could be driven to completion by heat. The NMR is consistent with the Diels-Alder Adduct, with HEA unsaturation peaks having completely disappeared. Note that 4 stereoisomer possibilities exist for the structure below.

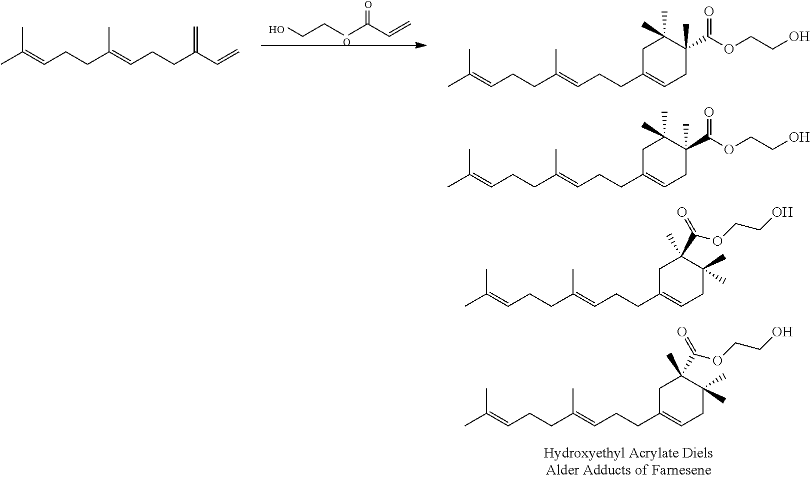

### Example 4

### Farnesene/Methyl Acrylate Diels-Alder Adduct

Methyl acrylate was added dropwise to beta-farnesene over a period of about one hour at 50° C. with no nitrogen purge. It was then heated with stirring at 70° C. for 3 days under these conditions. The yield of Diels-Alder adduct after 8 hours was 59.2% based on the ratio of the product Me ester peak to the methyl acrylate methyl peak by NMR.

The yield of Diels-Alder adduct after 3 days at 70° C. was 96.4% based on the ratio of the product Me ester peak to the methyl acrylate methyl peak by NMR.

### Example 5

### Preparation of Farnesene Maleate and MPEG Half Acid Ester Salt

Farnesene (25 g, 0.122 moles/98+% purity/Bedoukian) was mixed with maleic anhydride (MAH) (12.0 g/0.122 mole) in a 150 mL round bottom flask equipped with nitrogen pad, magnetic stirring and temperature control. The mixture was heated to 50° C., whereupon the reaction mixture turned orange and the MAH pellets dissolved. The reaction temperature rose to 202° C. over the course of approximately 1 minute, and as this exotherm was occurring; the mixture turned nearly colorless (very pale yellow). The reaction was cooled with an air gun to 115° C. A nitrogen bubbler was installed in place of the pad to encourage ring closure of any diacid to the anhydride.

The temperature was then raised to 140° C. and held for 30 min., at which point MPEG (mw=298/Stepan Company/36.4 g/0.122 mole) was added and allowed to react for another hour. Water (76.67 g/deionized) were added. The initial pH was 3.08. This was adjusted to 7.33 using 50% NaOH, to provide a liquid product that was pale yellow and had approximately 49% actives.

### Example 6

### Reaction of Farnesene Maleate with MTEG

| Component | Wt., g | mw | moles |
|---|---|---|---|
| Farnesene maleate | 99.15 | 302.4 | 0.3279 |
| Triethylene glycol | 54.91 | 164.2 | 0.3344 |
| mono methyl ether | | | |
| p-toluene sulfonic acid*H2O | 0.62 | 190.22 | 0.0033 |

The components listed in the chart were combined along with 250 mL of toluene in a 500 mL flask equipped with a Dean-Starke trap, condenser, heating mantle, thermocouple and magnetic stirring. After two hours of reflux, additional triethylene glycol mono methyl ether (MTEG, 54.91 g, 0.334 mole) was added and the reaction mixture was refluxed to remove water of reaction for 25 hours and allowed to cool overnight to room temperature. The toluene solution was extracted twice with 450 mL each of saturated sodium chloride solution made slightly basic with sodium hydroxide. The resulting toluene solution was evaporated by rotary evaporator to yield 180.9 g of product. Over the next several days, a salt precipitate formed in the product and settled to the bottom of the flask. Toluene was added to the reaction mixture to reduce the viscosity and enhance the precipitation of salt. This solution was then vacuum filtered through Celite 545 and the toluene removed using a rotary evaporator. The resulting product is di-(methyl ether triethylene glycol) farnesene maleate (100% actives).

### Example 7A

### Reaction of Farnesene Maleate with 3-(Dimethylamino)-1-Propylamine

| Component | Wt., g | mw | moles |
|---|---|---|---|
| Farnesene maleate | 99.15 | 302.4 | 0.3279 |
| N,N-Dimethyl amino | 33.50 | 102.18 | 0.3279 |
| propyl amine | | | |

The reactants above were combined with 250 mL of xylene in a 500 mL flask equipped with a Dean-Starke trap, condenser, heating mantle, thermocouple and magnetic stirring. Reflux was conducted with subsequent removal of water between the temperatures of 129° C. and 147° C. for 7 hours. The resulting product is farnesene maleimido dimethyl propylamine. Analysis suggests that the maleimide also contains farnesene maleamide half-acid.

### Example 7B

### Oxidation of Farnesene Maleimido Dimethyl Propylamine with Hydrogen Peroxide

| Component | Wt., g | mw | moles |
|---|---|---|---|
| Farnesene maleimide dimethyl | 110.00 | 386.58 | 0.28 |
| propylamine | | | |
| Water | 220.00 | | |
| 35% hydrogen peroxide | 28.19 | 34.00 | 0.29 |

The farnesene maleimide dimethyl propylamine made in Example 7A above and water, in the amounts listed in the above table, were placed in a 500 mL flask equipped with mechanical stirrer, condenser, addition funnel and heating mantel. The reaction mixture was heated to 53° C. followed by the addition of dry ice until the pH reached 6.95. Hydrogen peroxide was added drop-wise over the course of an hour with vigorous stirring until the oxidation was complete. The temperature was raised to 70° C. with stirring for 1.5 hours to complete the oxidation. The resulting product is DMAPA farnesene maleimide oxide. The actives content of this surfactant in water was measured at 32% by weight.

### Example 8A

### Reaction of Farnesene Maleate with MPEG 350

| Component | Wt., g | mw | moles |
|---|---|---|---|
| Farnesene maleate | 50.00 | 302.4 | 0.1653 |
| Triethylene glycol mono methyl ether | 27.15 | 164.2 | 0.1653 |
| MPEG 350 | 57.87 | 350 | 0.1653 |
| Toluene | 250.00 | | |
| p-toluene sulfonic acid*H2O | 0.31 | 190.22 | 0.0017 |

Triethylene glycol monomethyl ether was added to farnesene maleate along with toluene and heated to 100° C. for about 30 minutes in a 500 mL flask equipped with Dean-Starke trap, condenser, thermocouple, magnetic stirring, and heating mantle to permit ring opening and formation of the half acid ester. The monomethyl ether of polyethylene glycol (350 mw, MPEG 350) was added along with p-toluene sulfonic acid monohydrate and the reagents refluxed with subsequent azeotropic water removal for 4 days. The reactants were cooled to room temperature and sodium carbonate added with stirring for about 3 minutes, followed by vacuum filtration through celite 545 and rotary evaporation to remove the toluene. The resulting product is a diester of farnesene maleate having an actives content of 100%.

### Example 8B

### Reaction of Farnesene Maleate with MPEG 750

| Component | Wt., g | mw | moles |
|---|---|---|---|
| Farnesene maleate | 50.00 | 302.4 | 0.1653 |
| Triethylene glycol mono methyl ether | 27.15 | 164.2 | 0.1653 |
| MPEG 750 | 124.01 | 750 | 0.1653 |
| Toluene | 250.00 | | |
| p-toluene sulfonic acid*H2O | 0.31 | 190.22 | 0.0017 |

The triethylene glycol mono methyl ether was added to farnesene maleate along with toluene and heated to 100° C. for about 30 minutes in a 500 mL flask equipped with Dean-Starke trap, condenser, thermocouple, magnetic stirring, and heating mantle to permit ring opening and formation of the half acid ester. The monomethyl ether of polyethylene glycol (750 mw, MPEG 750) was added along with p-toluene sulfonic acid monohydrate and the reagents refluxed with subsequent azeotropic water removal for 4 days. The reactants were cooled to room temperature and sodium carbonate added with stirring for about 3 minutes, followed by vacuum filtration through celite 545 and rotary evaporation to remove the toluene. The resulting product is a diester of farnesene maleate, having an actives content of 100%.

### Example 9

### Evaluation of Surfactant Properties

Compositions are prepared, each of which contains one of the surfactants prepared in Examples 5 to 7 in water. The amount of surfactant added to water is dictated by the test procedure utilized to evaluate the surfactant properties. Foaming performance of each composition is tested by the shake foam test method as described above. Critical Micelle Concentration and Draves Wetting properties are also measured. The same tests are conducted on a composition containing Ammonyx LMDO in water, for comparison purposes. Ammonyx LMDO is a standard surfactant used in laundry, dishwash, hard surface cleaners and personal care applications to provide foam boosting and stabilization viscosity building and grease removal properties. The results are set forth below in Table 1.

TABLE 1

| | Example | Ammonyx | | |
|---|---|---|---|---|
| | 7B | LMDO | | |
| | DMAPA | Lauramido | | Example 5 |
| | Farnesene | Propyl | Example 6 | Sodium |
| | Maleimide | Dimethyl | DiMTEG | Farnesene |
| | Amine | Amine | Farnesene | Maleate |
| Chemical Name | Oxide | Oxide | Maleate | MPEG-12 |
| Critical Micelle | 17.7 | 88 | 55.4 | 190.4 |
| Concentration (mg/L) | | | | |
| Surface Tension as the | 31.8 | 27.9 | 35.3 | 34.4 |
| CMC (mNm) | | | | |
| Surface Tension at 10 | 42.5 | 45 | 44.2 | 52.2 |
| mg/L (mNm) | | | | |
| Shake Foam at 15 sec. | 337 | 307 | 135 | 212 |
| (mL) | | | | |
| Shake Foam at 5 min. | 337 | 285 | 122 | 126 |
| (mL) | | | | |
| Shake Foam with Castor | 212 | 162 | 117 | 196 |
| Oil at 15 sec. (mL) | | | | |
| Shake Foam with Castor | 212 | 162 | 115 | 102 |
| Oil at 5 min. (mL) | | | | |
| Draves Wetting (sec.) | 8.5 | 37 | 14 | 29 |

From the results, it can be seen that all of the surfactants derived from farnesene maleate provided better wetting than the lauramido propyl dimethyl amine oxide surfactant. Such wetting properties are useful in applications such as agricultural pesticide compositions to provide faster wetting which can lead to faster kill times for the pesticides. Such wetting properties are also useful in applications such as laundry detergent compositions since they may provide better penetration of detergents for soil removal. The results also demonstrate that DMAPA farnesene maleimide oxide provides more favorable critical micelle concentration and better foaming than lauramido propyl dimethyl amine oxide.

### Example 10

### Evaluation of Surfactant Properties

Compositions are prepared that contain each of the surfactants prepared in Examples 8A and 8B in water. Foaming performance of each composition is tested by the shake foam test method as described above. Critical Micelle Concentration and Draves Wetting properties are also measured. For comparison purposes, the same tests are conducted on a composition containing BIOSOFT 25-7, available from Stepan Co., Northfield, Ill., in water, and on a composition containing Makon TD-18, available from Stepan Co., Northfield, Ill. BIOSOFT 25-7 is an ethoxylated alcohol having a Hydrophilic Lipophilic Balance (HLB) of 12, which is comparable to the HLB of farnesene maleate diethoxylate prepared in Example 8A. Makon TD-18 is a tridecyl ethoxylated alcohol having an HLB of 16, which is comparable to the HLB of farnesene maleate diethoxylate prepared in Example 8B. The results are shown below in Table 2.

TABLE 2

| | | Example 8A | | Example 8B |
|---|---|---|---|---|
| | Biosoft 25-7 | Farnesene | Makon TD-18 | Farnesene |
| | C12-C15 Alcohol | Maleate | Tridecyl Alcohol | Maleate |
| | Ethoxylated with | Diethoxylate | Ethoxylated with | Diethoxylate |
| Chemical Name | 7 EO/HLB = 12 | HLB = 12 | 18 EO/HLB = 16 | HLB = 15 |
| Critical Micelle | 6.3 | 102.4 | 260.1 | 142.7 |
| Concentration (mg/L) | | | | |
| Surface Tension at the | 31.1 | 33.7 | 31.3 | 33.5 |
| CMC (mNm) | | | | |
| Surface Tension at 10 | 30.1 | 46 | 48.4 | 47.9 |
| mg/L (mNm) | | | | |
| Shake Foam at 15 sec. | 255 | 202 | 300 | 215 |
| (mL) | | | | |
| Shake Foam at 5 min. | 247 | 130 | 157 | 140 |
| (mL) | | | | |
| Shake Foam with Castor | 245 | 205 | 252 | 202 |
| Oil at 15 sec. (mL) | | | | |
| Shake Foam with Castor | 175 | 115 | 165 | 127 |
| Oil at 5 min. (mL) | | | | |
| Draves Wetting (sec.) | 12.5 | 1.5 | 48.5 | 16.5 |
| Acid Value (mg KOH/g of | Nm | 13.1 | Nm | 14.6 |
| sample) | | | | |
| pH | 5.0 | 4.0 | 5.0 | 4.0 |

From the results, it can be seen that each of the surfactants derived from farnesene maleate has better wetting properties than a conventional ethoxylated fatty alcohol surfactant with comparable HLB.

### Example 11

### Evaluation of Cleaning Performance of DMAPA Farnesene Maleimide Oxide

Test samples were prepared containing 300 g of a 0.2% actives solution of the following formulations.

| | | Amount | D.I. | |
|---|---|---|---|---|
| Test Sample | % Actives | Added (g) | Water | Total |
| Example 7B DMAPA Farnesene | 32.00 | 1.88 | 98.13 | 100.00 |
| Maleimide Oxide | | | | |
| Ammonyx LO ® | 30.10 | 1.99 | 98.01 | 100.00 |
| Ammonyx LMDO ® | 32.72 | 1.83 | 98.17 | 100.00 |

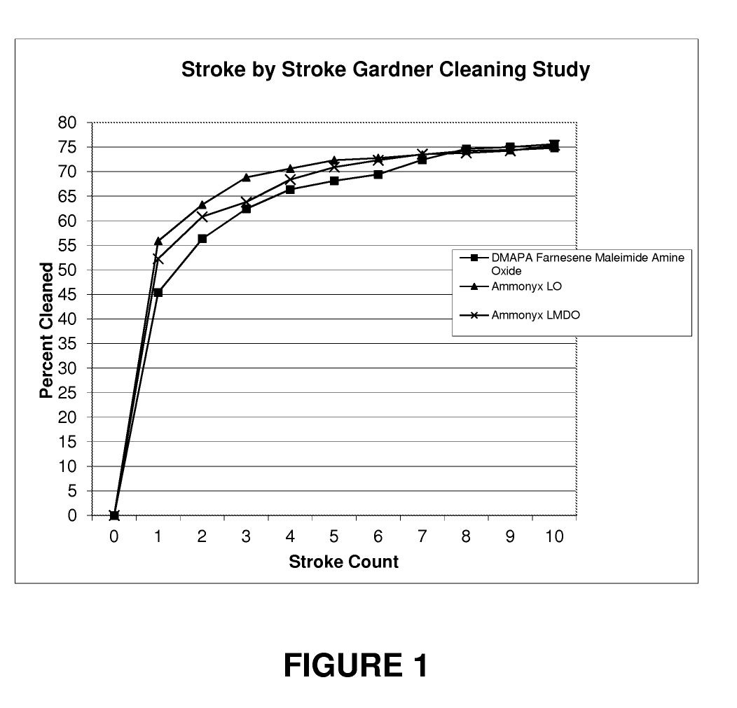

The test samples were evaluated for cleaning performance using the Gardner Cleaning Test method described above. The results are set forth below in Table 3 and shown graphically in FIG. 1.

TABLE 3

| | Average Percent Clean | | | | | | | | | |
|---|---|---|---|---|---|---|---|---|---|---|
| | Stroke Number | | | | | | | | | |
| Product | 1 | 2 | 3 | 4 | 5 | 6 | 7 | 8 | 9 | 10 |
| DMAPA | 45.36 | 56.33 | 62.42 | 66.36 | 68.12 | 69.43 | 72.39 | 74.63 | 75.00 | 75.60 |
| Farnesene | | | | | | | | | | |
| Maleimide Oxide | | | | | | | | | | |
| Ammonyx LO | 55.87 | 63.25 | 68.83 | 70.62 | 72.34 | 72.76 | 73.46 | 74.26 | 74.40 | 74.82 |
| Ammonyx LMDO | 52.25 | 60.80 | 63.80 | 68.37 | 70.90 | 72.31 | 73.55 | 73.79 | 74.24 | 75.35 |
| | Standard Deviation Among 3 Replicate Tiles | | | | | | | | | |
| | Stroke Number | | | | | | | | | |
| Product | 1 | 2 | 3 | 4 | 5 | 6 | 7 | 8 | 9 | 10 |
| DMAPA | 6.10 | 6.03 | 5.33 | 6.44 | 5.57 | 4.62 | 3.38 | 1.87 | 1.42 | 1.19 |
| Farnesene | | | | | | | | | | |
| Maleimide Oxide | | | | | | | | | | |
| Ammonyx LO | 1.20 | 2.32 | 2.06 | 1.76 | 2.69 | 2.52 | 3.27 | 3.89 | 3.96 | 4.29 |
| Ammonyx LMDO | 5.12 | 4.69 | 3.02 | 3.22 | 3.21 | 3.66 | 3.26 | 2.87 | 3.19 | 3.20 |

The results show that the experimental sample in accordance with the present technology, containing the Example 7B DMAPA farnesene maleimide oxide, gave cleaning performance comparable to Ammonyx LMDO, which is a high performance amine oxide commercially used for liquid dish detergents.

### Example 12

### Preparation of Diphenoxyethyl Farnesene Maleate 1.3 mole Sulfonate

Farnesene maleate (126.1 g, 0.417 mole, previously prepared) was mixed with 2-phenoxy ethanol (350 g, 2.533 moles) and heated to 140° C. to ring open the anhydride. Tetra(n-butyl) titanate (0.22 g) was added at this temperature and the reaction heated to 200° C. with nitrogen purge. Butylated hydroxytoluene (0.1 g) was added to preserve the color. Upon reaching 195° C., the heat was turned off and the reaction allowed to sit overnight under nitrogen. The next morning, the heat was reinitiated and the nitrogen rate turned up to provide a purge. Note that a gas dispersion tube was used for introduction of nitrogen into the reaction mixture within the vessel. Condensate (166 g) was collected and discarded. Note that due to the rate of nitrogen purge, substantial amounts of 2-phenoxylethanol were emitted as vapor from the reaction through the condenser. The product color was amber and transparent. The weight of the additional condensate at this point was 16.1 g. The acid value of the product was 3.2 mg KOH/g while the OHV was 4.4 mg KOH/g. The theoretical percentage by weight of 2-phenoxyethanol based on this OHV is 1.08%.

Diphenoxyethyl farnesene maleate (210 g, 375 mmol) was dissolved in ethyl acetate (400 mL) in a Parr shaker bottle and 10% palladium on carbon (20 g) was added. The bottle was sealed, pressurized with H2 (g) to 50 psi, and allowed to react at room temperature for 24 hours. Periodically, aliquots were removed, filtered through a small plug of Celite, and concentrated in vacuo. The samples were used for proton

NMR to monitor the reaction. When complete, the mixture was filtered through Celite, washed with ethyl acetate, and concentrated in vacuo to dryness to give a pale yellow oil (208 g, 98%).

In a small scale batch reactor maintained at 25° C. via the circulation of thermostated water through the reactor jacket and with a pre-established 2 L/m flow of nitrogen through the fritted bottom of the reactor, 60.48 g (0.107 mol) of diphenoxyethyl farnesene maleate was added to 50 mL of methylene chloride. Over a 30 minute period, 11.21 g (0.14 mole) of sulfur trioxide was evaporated via a 140° C. flash-pot and was bubbled through the batch reactor using the 2 L/m nitrogen stream. The addition rate of SO3 was such that the reaction temperature never exceeded 30° C. During the addition of SO3 another 50 mL of methylene chloride was added to the reaction. At the end of the addition, the reaction was maintained for an additional 5 minutes and was then transferred to a round bottom flask and placed under vacuum for ˜1 hour. Titration with cyclohexylamine showed 1.25% sulfuric acid and 85.87% sulfonic acid. The sample was then titrated for total acid and from these results the acid was neutralized using 89.7 g of water and 9.8 g of 50% NaOH (aq) to give diphenoxyethyl farnesene maleate 1.3 mole sulfonate.

This anionic surfactant (10 wt. % actives) was mixed with 20 wt. % Neodol® 25-7 (C12-C15 fatty alcohol 7 mole ethoxylate) actives, and 30 wt. % actives Steol® CS-370 (sodium laur(3)eth sulfate) in water to provide a primary surfactant blend having a viscosity of 6509 cps at 25° C. A control prepared using 20 wt. % actives Neodol® 25-7 and 40 wt. % actives Steol® CS-370 yielded a viscosity of 50,280 cps at 25° C. These results clearly indicate the utility of diphenoxyethyl farnesene maleate 1.3 mole sulfonate to facilitate pourable, high concentration laundry detergent formulations.

### Example 13

### Preparation of Ammonium N-Ethyl Farnesene Maleimide Sulfate

| Component | Wt.,g | mw | moles |
|---|---|---|---|
| Farnesene | 67.00 | 204.35 | 0.327869 |
| Maleic Anhydride | 32.1598 | 98.06 | 0.327869 |
| Ethanolamine (bp = 170° C.) | 19.6361 | 61.08 | 0.321311 |

The reaction between farnesene and maleic anhydride was conducted as previously described in Example 5, using the quantities of farnesene and maleic anhydride shown in the table above, in a 250 mL, round bottom flask. The reaction was allowed to cool to room temperature. Ethanol amine was added drop-wise with stirring, resulting in an exotherm to about 140° C. Stirring was continued, and the reaction mixture was maintained at 130° C. for one hour, then nitrogen purge begun and the mixture heated to 175° C. with this purge for 6 hours. A short section of Tygon hose was vented downward into a beaker to collect the water and permit venting of steam, and near the end, condensate in the adapters and necks of the flask was driven off with a heat gun. The product provided an NMR consistent with the pure maleimide and the reaction scheme provided below

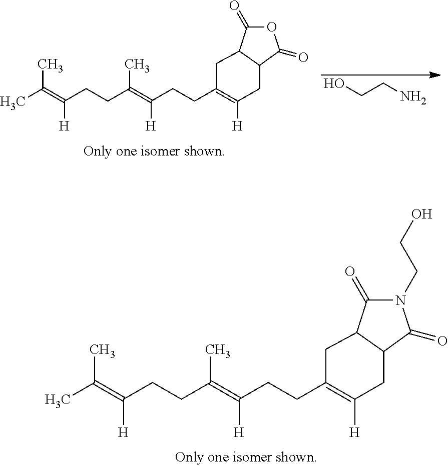

The hydroxyethyl farnesene maleimide was mixed with sulfamic acid in the quantities indicated in the chart below in a 250 mL, 3 neck flask equipped with nitrogen purge, heating mantle, magnetic stirring, and thermocouple temperature control. The mixture was heated to 100° C. for 17 hours.

| Sulfation Step Components | Wt., g | mw | moles |
|---|---|---|---|
| N-Hydroxyethyl Farnesene Maleimide | 75.00 | 345.49 | 0.217083 |
| Sulfamic Acid | 23.19 | 97.1 | 0.238791 |

wt. sub total = 98.19

| Neutralization Step Components | Wt., g | mw | moles |
|---|---|---|---|
| Ethanol Amine | 1.33 | 61.08 | 0.021708 |
| Methanol | 118.77 | 32.04 | 0.023879 |

Proton NMR indicated a poor yield of sulfation, therefore, 75 mL of dimethyl formamide were added and the reaction stirred with heating and nitrogen pad overnight at 120° C. After this step, proton NMR indicated an essentially complete conversion to sulfate, represented by the drawing below.

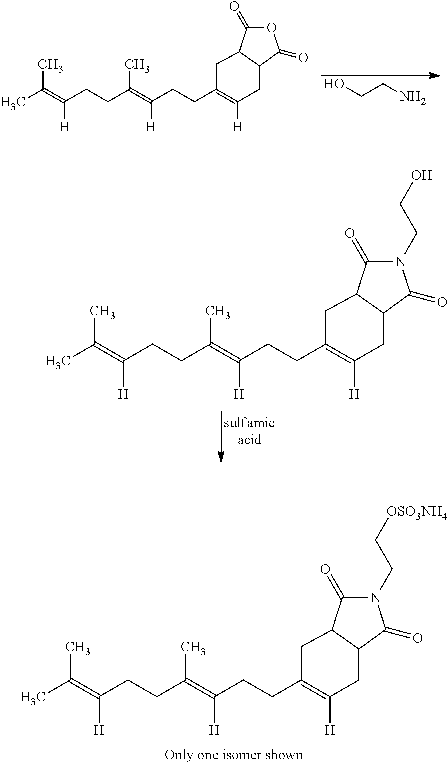

Ethanol amine (amt. indicated in the chart above) was then added to this reaction product to neutralize the excess sulfamic acid, along with 150 mL of methanol at 55° C. to enhance fluidity. Manual stirring with a spatula was initially required, due to the taffy-like viscosity at this temperature. After 15 minutes of stirring and homogenization, the pH was 7.73. The resulting solution was stripped of methanol via rotary evaporator to provide ammonium N-ethyl farnesene maleimide sulfate.

The surfactant properties for this material were measured and are provided in Table 4 below. The Draves wetting for this surfactant at 12.5 seconds indicates potential utility in applications such as laundry, cleaning and agricultural adjuvants.

### Example 14

### Hydroxyethyl Acrylate/Farnesene Diels Alder Adduct

Hydroxyethyl acrylate was added dropwise to beta-farnesene over a period of about one hour at 50° C. with no nitrogen pad or purge. The exotherm was minimal, so the temperature was raised over an hour to 180° C. for 6 hours. The resulting proton NMR is consistent with the Diels Alder adduct provided in Diels Alder Reaction Scheme E.

### Example 15

### Ammonium Ethyloxy Farnesene Acrylate Sulfate

Farnesene/hydroxyethyl acrylate Diels-Alder adduct (90.87 g/mw=320.47/0.284 mole/3838-29-6) was added to sulfamic acid (30.3 g/0.312 mole) whereupon the mixture was heated to 100° C. under nitrogen purge over the course of 15 hours. HNMR indicated no loss of CH2 hydrogens adjacent to the OH. C13NMR indicated poor conversion.

In light of the poor conversion, and the observation that a large portion of crystalline sulfamic acid remained in the flask, dimethyl formamide (75 mL) was added and the reaction stirred with heating overnight at 120° C. The dimethyl formamide was removed by purging vigorously with nitrogen for 4 hours at 120° C. Ethanol amine (1.71 g, 0.028 mole) was then added along with 150 mL of methanol. The methanol was rapidly added with stirring at 120° C., and no manual stirring was required to achieve dissolution during cooling to 55° C. The pH was 7.65 at this temperature. The resulting solution was filtered through Celite 545 and stripped of methanol via rotary evaporator to provide ammonium ethyloxy farnesene acrylate sulfate as illustrated by the scheme below.

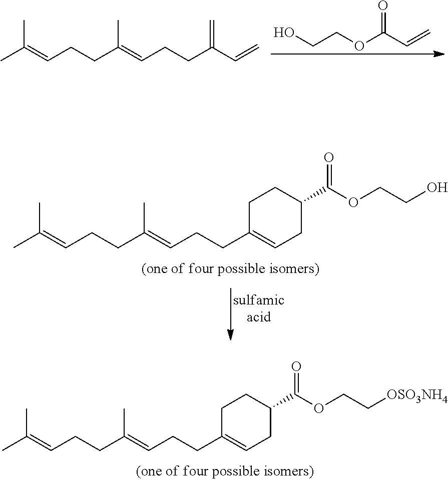

The surfactant properties for this material were measured and are provided in Table 4 below. Although the CMC for this surfactant was very high, the hydrotropic product provided compaction performance when used at 10% actives for a 60% total actives laundry detergent blend containing 10% actives Neodol® 25-7, 40% actives Steol® CS-370 and 10% actives ammonium ethyloxy farnesene acrylate sulfate, yielding a flowable (viscosity of 9239 cps) formulation at 25° C. In contrast, a control formula containing 50% actives Steol® CS-370 and 10% actives Neodol® 25-7 provided a non-flowable formulation having a viscosity of 50,280 cps at 25° C. This clearly demonstrates the utility of this surfactant to enable flowable, highly concentrated laundry detergent formulations. Additionally, the Draves wetting for this surfactant at 10 seconds indicates potential utility in applications such as laundry, cleaning and agricultural adjuvants.

TABLE 4

| | Ammonium Ethyloxy | Ammonium N-Ethyl |
|---|---|---|
| | Farnesene Acrylate | Farnesene Maleimide |
| Chemical Name | Sulfate (Example 15) | Sulfate (Example 13) |
| Critical Micelle Concentration (mg/L) | >10,000 | 1047 |
| Surface Tension at the CMC (mNm) | NA | 31.9 |
| Surface Tension at 10 mg/L (mNm) | 68.9 | 64.9 |
| Shake Foam at 15 sec. (mL) | 292 | 312 |
| Shake Foam at 5 min. (mL) | 282 | 295 |
| Shake Foam with Castor Oil at 15 sec. (mL) | 177 | 300 |
| Shake Foam with Castor Oil at 5 min. (mL) | 170 | 295 |
| Draves Wetting (sec.) | 10 | 12.5 |
| Actives (%) | 95% | 95% |

The present technology is now described in such full, clear, and concise terms as to enable a person skilled in the art to which it pertains, to practice the same. It is to be understood that the foregoing describes preferred embodiments of the present technology and that modifications may be made therein without departing from the spirit or scope of the present technology as set forth in the claims.

What is claimed is:

1. A composition comprising a cationic, amine oxide, amphoteric, anionic, or nonionic surfactant which is the reaction product of
(i) a Diels Alder adduct formed from:
(a) farnesene or myrcene or mixtures thereof; and
(b) a dienophile selected from the group consisting of maleic anhydride, itaconic anhydride, dimethyl maleate, dimethyl itaconate, maleic acid, itaconic acid, fumaric acid, dimethyl fumarate, acrylic acid, methyl acrylate, ethyl acrylate, butyl acrylate, methyl methacrylate, methacrylic acid, benzaldehyde, hydroxyethyl acrylate, hydroxyethyl methacrylate, hydroxypropyl acrylate, hydroxypropyl methacrylate, acrylonitrile, acrylamide, N-hydroxyethyl maleimide, maleimide, vinyl alkyl ketones, methacrolein, and mixtures thereof; reacted with
(ii) at least one derivatizing agent selected from the group consisting of:
mono-alkyl ether of polyethylene glycol, mono-alkyl ether of polypropyloxy-polyethyloxy block copolymer, mono-alkyl ethers of polybutyloxy-polyethyloxy block copolymer, amine-containing alkylene glycol, amine-containing polyalkylene glycol, mono- or oligo-alkylene glycol, amine, polyamine, aliphatic alcohol, cycloaliphatic sultone, aromatic substituted alcohol, hydrogenating agent, aryloxy alkylene alcohol, sulfonating agent, phosphating agent, sulfating agent, aryl-alkyl halide, dimethyl sulfate, epichlorohydrin, and combinations thereof.

2. The composition of claim 1, wherein the dienophile is maleic anhydride, itaconic anhydride, dimethyl maleate, dimethyl itaconate, maleic acid, itaconic acid, fumaric acid, dimethyl fumarate, acrylic acid, methyl acrylate, ethyl acrylate, butyl acrylate, methyl methacrylate, methacrylic acid, or mixtures thereof, and
the at least one derivatizing agent is selected from mono-alkyl ether of polyethylene glycol, mono-alkyl ether of polypropyloxy-polyethyloxy block copolymer, mono-alkyl ether of polybutyloxy-polyethyloxy block copolymer, and mixtures thereof, wherein the derivatizing agent comprises from one to about 30 moles of ethylene oxide and the alkyl ether group contains from 1 to 6 carbons.

3. The composition of claim 1, wherein the dienophile is maleic anhydride, itaconic anhydride, dimethyl maleate, dimethyl itaconate, maleic acid, itaconic acid, fumaric acid, dimethyl fumarate, acrylic acid, methacrylic acid, methyl acrylate, methyl methacrylate, or mixtures thereof, and
the at least one derivatizing agent comprises the combination of at least one aromatic alcohol, a hydrogenating agent, and at least one sulfonating agent.

4. The composition of claim 3, wherein the aromatic alcohol is an aromatic substituted hydroxy alkane.

5. The composition of claim 4, wherein the sulfonating agent is sulfur trioxide or a derivative thereof.

6. The composition of claim 1, wherein the dienophile is acrylonitrile, and
the derivatizing agent comprises the combination of at least one hydrogenating agent, at least one aliphatic alcohol, and at least one alkyl halide, aryl alkyl halide, epichlorohydrin, or dimethyl sulfate.

7. The composition of claim 6, wherein the at least one hydrogenating agent is hydrogen.

8. The composition of claim 6, wherein the at least one aliphatic alcohol is methanol.

9. The composition of claim 6, wherein the at least one alkyl halide is methyl chloride.

10. The composition of claim 6, wherein the at least one aryl alkyl halide is benzyl chloride.

11. A composition comprising a solvent which is the reaction product of
(i) a Diels Alder adduct formed from:
(a) farnesene, myrcene or mixtures thereof; and
(b) a dienophile selected from the group consisting of maleic anhydride, itaconic anhydride, dimethyl maleate, dimethyl itaconate, maleic acid, itaconic acid, fumaric acid, dimethyl fumarate, acrylic acid, methyl acrylate, ethyl acrylate, butyl acrylate, methyl methacrylate, methacrylic acid, and mixtures thereof; reacted with
(ii) at least one derivatizing agent selected from the group consisting of monoalkyl ethers of polyethylene glycols, mono-alkyl ethers of polypropyloxy-polyethyloxy block copolymers, mono-alkyl ethers of polybutyloxy-polyethyloxy block copolymers, aliphatic alcohols, aromatic alcohols, aryloxy alkylene alcohols, hydrogenating agents, alkyl halides, aryl-alkylhalides, and combinations thereof.

12. The composition of claim 11, wherein the dienophile is maleic anhydride, itaconic anhydride, dimethyl maleate, dimethyl itaconate, maleic acid, itaconic acid, fumaric acid, dimethyl fumarate, acrylic acid, methacrylic acid, methyl acrylate, ethyl acrylate, butyl acrylate, methyl methacrylate, or mixtures thereof, and
the at least one derivatizing agent is selected from monoalkyl polyethylene glycol, mono-alkyl ethers of polypropyloxy-polyethyloxy block copolymers, mono-alkyl ethers of polybutyloxy-polyethyloxy block copolymers, and mixtures thereof.

13. The composition of claim 11, wherein the dienophile is maleic anhydride, itaconic anhydride, dimethyl maleate, dimethyl itaconate, maleic acid, itaconic acid, fumaric acid, dimethyl fumarate, acrylic acid, methacrylic acid, methyl acrylate, ethyl acrylate, butyl acrylate, methyl methacrylate, or mixtures thereof, and
the at least one derivatizing agent is at least one aromatic or aryloxy alkylene alcohol.

14. The composition of claim 11, wherein the aromatic or aryloxy alkylene alcohol is an aromatic substituted hydroxy alkane.

15. A process for producing a surfactant or solvent derived from a Diels-Alder adduct, comprising the steps of:
(a) forming a Diels-Alder adduct by reacting farnesene, myrcene, or mixtures thereof with at least one dienophile selected from the group consisting of maleic anhydride, itaconic anhydride, dimethyl maleate, dimethyl itaconate, maleic acid, itaconic acid, fumaric acid, dimethyl fumarate, acrylic acid, methacrylic acid, methyl acrylate, ethyl acrylate, butyl acrylate, methyl methacrylate, acrylonitrile, acrylamide, and mixtures thereof; and
(b) reacting the Diels-Alder adduct with at least one derivatizing agent selected from the group consisting of:
mono-alkyl ether of polyethylene glycol, mono-alkyl ether of polypropyloxy-polyethyloxy block copolymer, mono-alkyl ethers of polybutyloxy-polyethyloxy block copolymer, mono- or oligo-alkylene glycol, amine, polyamine, aliphatic alcohol, cycloaliphatic sultone, aromatic substituted alcohol, hydrogenating agent, aryloxy alkylene alcohol, sulfonating agent, phosphating agent, sulfating agent, alkyl halide, aryl-alkyl halide, dimethyl sulfate, epichlorohydrin, and combinations thereof.

16. The process of claim 15, wherein the Diels-Alder adduct is reacted with monoalkyl polyethylene glycol, mono-alkyl ether of polypropyloxy-polyethyloxy block copolymer, mono-alkyl ether of polybutyloxy-polyethyloxy block copolymer, or mixtures thereof to form an ester of the Diels-Alder adduct.

17. The process of claim 15, wherein the Diels-Alder adduct is reacted with monoalkyl polyethylene glycol, mono-alkyl ether of polypropyloxy-polyethyloxy block copolymer, mono-alkyl ether of polybutyloxy-polyethyloxy block copolymer, or mixtures thereof in equimolar amounts, and the reaction product is further reacted with a base.

18. The process of claim 15, wherein the Diels-Alder adduct is reacted with at least one aromatic or aryloxy alkylene alcohol to form an intermediate ester reaction product, and the intermediate ester reaction product is further reacted with a hydrogenating agent and at least one sulfonating agent.

19. The process of claim 18, wherein the at least one aromatic or aryloxy alkylene alcohol is 2-phenoxyethanol, benzyl alcohol, 2-phenyl ethanol, an ethoxylated phenol, or a mixture thereof.

20. The process of claim 15, wherein the Diels-Alder adduct is formed from farnesene, myrcene, or mixtures thereof and acrylonitrile, and the adduct is reacted with at least one hydrogenating agent, and further reacted with at least one aliphatic alcohol and at least one alkyl or aryl halide or epichlorohydrin or dimethyl sulfate or a mixture thereof.

21. The process of claim 20, wherein the hydrogenating agent is hydrogen gas.

22. The process of claim 20, wherein the at least one aliphatic alcohol is methanol.

23. The process of claim 20, wherein the at least one alkyl halide is methyl chloride.

24. The process of claim 20, wherein the at least one aryl halide is benzyl chloride.

25. A composition comprising a nonionic surfactant which is the reaction product of a Diels Alder adduct formed from:
(a) farnesene or myrcene or mixtures thereof; and
(b) a dienophile containing a monoalkyl ether of polyethylene glycol, a monoalkyl ether of a polypropyloxy-polyethyloxy block copolymer, or a polyalkylene glycol containing ethylene oxide, propylene oxide, butylene oxide, or mixtures thereof.

26. A process for producing a surfactant derived from a Diels-Alder adduct comprising the steps of:
(i) derivatizing (a) a dienophile selected from the group consisting of maleic anhydride, itaconic anhydride, dimethyl maleate, dimethyl itaconate, maleic acid, itaconic acid, fumaric acid, dimethyl fumarate, acrylic acid, methyl acrylate, ethyl acrylate, methyl methacrylate, methacrylic acid, benzaldehyde, hydroxyethyl acrylate, hydroxyethyl methacrylate, hydroxypropyl acrylate, hydroxypropyl methacrylate, acrylonitrile, acrylamide, N-hydroxyethyl maleimide, maleimide, and mixtures thereof; with (b) at least one derivatizing agent selected from the group consisting of monoalkyl ether of polyethylene glycol, mono-alkyl ether of polypropyloxy-polyethyloxy block copolymer, amine-containing monoalkyleneglycol, amine-containing polyalkyleneglycol, mono-alkyl ethers of polybutyloxy-polyethyloxy block copolymer, polyalkylene glycols containing ethylene oxide, propylene oxide, butylene oxide or mixtures thereof, alkylene glycol, an alkylene oxide containing 2-4 carbon atoms, amine, polyamine, aliphatic alcohol, aromatic alcohol, aryloxy alkylene alcohol, sulfonating agent, sulfating agent, oxidizing agent, sugars, alkyl halide, aryl-alkylhalide, dimethyl sulfate, and combinations thereof to form a derivatized dienophile; and
(ii) reacting the derivatized dienophile with farnesene or myrcene or mixtures thereof by a Diels-Alder reaction.
